(19) United States
(12) Patent Application Publication
(10) Pub. No.: US 2017/0240854 A1
(43) Pub. Date: Aug. 24, 2017

(54) A BIOREACTOR MODULE, A BIOREACTOR SYSTEM AND METHODS FOR THICK TISSUE SEEDING AND CULTIVATION IN AN HIREARCHICAL ORGANIZATION AND PHYSIOLOGICAL MIMIKING CONDITIONS

(71) Applicants: Nanyang Technological University, Singapore (SG); TECHNION RESEARCH AND DEVELOPMENT FOUNDATION LTD, Haifa (IL)

(72) Inventors: Marcelle MACHLUF, Haifa (IL); Udi SARIG, Singapore (SG); Maskit GVIRTZ, Haifa (IL); Subbu VENKATRAMAN, Singapore (SG)

(21) Appl. No.: 15/513,907

(22) PCT Filed: Sep. 23, 2015
(86) PCT No.: PCT/SG2015/050339

§ 371 Date: Mar. 23, 2017

(30) Foreign Application Priority Data
Sep. 23, 2014 (SG) 10201405985S

(51) Int. Cl. C12M 3/00; C12M 1/42; C12M 1/12; C12N 5/071; C12M 1/00

(52) U.S. Cl. CPC C12M 21/08; C12N 5/069; C12N 5/0602; C12M 23/22; C12M 25/14; C12M 35/02; C12M 35/04; C12M 29/10; C12N 2533/50; C12N 2501/165; C12N 2501/115; C12N 2502/28; C12N 2502/00

(57) ABSTRACT

The invention provides a bioreactor and methods for tissue cultivation. A bioreactor module comprises a container, a holder adapted to hold a scaffold containing an inherent vascular network, an inlet connectable to a vessel of the inherent vascular network, an inflatable device disposed within the container, and a pair of electrodes attached to opposing walls of the container, wherein the holder is removably receivable in the container and the inflatable device has a conduit extending through a wall of the container. An alternative embodiment provides an in-vitro method for tissue cultivation, comprising seeding an interior and an exterior of a vessel of an inherent vascular network of a scaffold with a first and a second cell type, respectively, and perfusing through the inherent vascular network with culture medium to facilitate compartmentalized co-cultivation of the first and the second cell type in different niches of the tissue. A further embodiment provides an in-vitro method for tissue cultivation, comprising seeding a surface of a scaffold with a predetermined cell type, and perfusing the scaffold with culture medium from an opposite surface of the scaffold through the scaffold and towards the seeded surface to create a nutrient/oxygen gradient and cause migratory diffusion induced penetration of cells towards the opposite surface.

### CROSS-REFERENCE TO RELATED APPLICATIONS

The present application claims the benefit of the Singapore patent application No. 10201405985S filed on 23 Sep. 2014, the entire contents of which are incorporated herein by reference for all purposes.

### TECHNICAL FIELD

Embodiments relate generally to a bioreactor module, a bioreactor system and methods for tissue cultivation.

### BACKGROUND

Tissue Engineering (TE) and Regenerative Medicine (RM) are interdisciplinary fields combining knowledge of cell and tissue biology, material science, biomedical engineering and clinical medicine aiming to promote endogenous regeneration of terminally damaged and currently unrepairable tissues, and/or develop substitute tissues to restore overall organ function. Those substitute tissues are fabricated by combining various living cells, either terminally differentiated or of progenitor/stem-cell characteristics, with/without scaffolding biomaterials that provide support and relevant biochemical cues guiding tissue development.

Some documentation exists describing either whole organ TE templates on the one hand, or simple tissue types TE of diffusion limited dimensions and biological functionalities on the other hand. Both alternatives lack applicability when referring to engineering of partial replacement tissues of human physiological relevancy.

The use of whole organ templates relies on preserved intact vascular networks of the whole organ for effective recellularization, and requires vast cell quantities, and compartmentalized cellular organization complexity within the organ, which are currently unachievable using reported technologies. As such, documents relating to whole organ template recellularization may be general and may not be backed up, in most cases, with supportive technological evidence of human relevant densities, and compartmentalized successful cellularization.

On the other hand, to scale-up a bioreactor for small tissue segments to support TE for larger physiologically applications, such as those required for therapeutic applications, are not trivial modifications nor do the forces at play scale-up in a linear manner. Hence grave challenges exist when aiming to achieve larger scales while enabling effective recellularization, maintaining convection of nutrients and oxygen, removal of waste products and sustaining tissue and construct viabilities for long term (>20 days). Major challenges relate to harsh gradients in terms of osmolarity and nutrient supply and to diffusion limitations particularly of oxygen supply under static culture conditions, which in soft tissues is defined as ˜100 μm. Therefore several approaches were attempted using either forced perfusion through porous scaffolds, or the development of vascular network within the developing tissue. Forced media perfusion may achieve some degree of increased cell penetrance and viabilities, however, it is limited due to biological boundaries of shear stresses and pressure regimes employed. The ultimate thicknesses achieved under dynamic culture conditions (<600 μm), are still far from that of the natural left ventricular wall (˜10-15 mm). This value probably represents the upper bound of this approach, due to a tradeoff between insufficient supply of too-low perfusion pressures and excessive shear stress jeopardizing cell viabilities when too-high pressures are employed. On the other hand, the development of vascular network in parallel to cell driven tissue development is a considerable long process even in the uterus containing a developing embryo (which could be considered as the best bioreactor system). Currently, no known bioreactor system is able to provide long term (>20 days) supportability for ex vivo dynamic cultivation of tissue construct with size suitable for clinical use.

Further, despite major advancements in the fields of biomaterials and cell biology, limited success has been reported in producing clinically relevant tissue-engineered constructs. For example, in cardiac regeneration following myocardial infarction, there is limited success regardless of the material type or cell delivery platform used (i.e., patch or injection based). The clinical application of existing solutions is limited by the lack of functional vascularization, and/or the inability to ensure efficient cell support in clinically relevant thick tissue constructs.

Consequently, however encouraging the data published to date may be, the lack of a connectable vascular tree during transplantation has led to a long lag time while angiogenesis occurs, speculated to result in minimal cell retention in the heart's harsh environment. Vascularization is, therefore, needed both to support the establishment of ex vivo cultivated cell-seeded constructs, and to provide a connectable vascular tree that can instantly supply the tissue upon transplantation.

Functional vascular supply is one of the most crucial impediments determining the post-transplantational fate of recellularized tissue constructs. Several strategies were suggested to circumvent these limitations. The use of cocultures incorporating endothelial and pericyte-like cells, with or without parenchymal model cells, was shown to improve the prospects of statically cultivated constructs by enhancing post-transplantation vessel sprouting and connectivity to the host tissue. In these strategies, the key hurdle to achieving ultimate human applicable sized grafts is the long lag-time required for functional angiogenesis to occur (˜2-3 weeks post-implantation).

Therefore, there is a need to address some of the issues discussed above in relation to the existing apparatus and methods for tissue cultivation.

### SUMMARY

According to various embodiments, there is provided a bioreactor module including a container; a holder removably receivable in the container, the holder adapted to hold a scaffold containing an inherent vascular network; an inlet connectable to a vessel of the inherent vascular network of the scaffold; an inflatable device disposed within the container, the inflatable device having a conduit extending through a wall of the container; and a pair of electrodes attached to opposing walls of the container.

According to various embodiments, there is provided a bioreactor system including a bioreactor module as substantially described herein; a mechanical stimulation subsystem adapted to control an inflatable device of the bioreactor module to generate mechanical stimulation by controlling the inflation of the inflatable device; and an electrical stimulation subsystem adapted to control a pair of electrodes of the bioreactor module to generate electrical stimulation by transmitting electric pulses from the pair of electrodes.

According to various embodiments, there is provided an in-vitro method for tissue cultivation, including: connecting a vessel of an inherent vascular network of a scaffold to an inlet of a bioreactor module as described herein; and perfusing the scaffold via the inlet of the bioreactor module.

According to various embodiments, there is provided an in-vitro method for tissue cultivation, including seeding an interior of a vessel of an inherent vascular network of a scaffold with a first cell type; seeding an exterior surface of the scaffold with a second cell type; and perfusing through the inherent vascular network of the scaffold with culture medium to facilitate compartmentalized co-cultivation of the first cell type and the second cell type in different niches of the tissue.

According to various embodiments, there is provided an in-vitro method for tissue cultivation, including seeding a surface of a scaffold with a predetermined cell type; and perfusing the scaffold from an opposite surface of the scaffold through the scaffold and towards the seeded surface with culture medium to provide flow of nutrients and oxygen through the the scaffold to create a nutrient/oxygen gradient between the opposite surface and the seeded surface of the scaffold to cause migratory diffusion induced penetration of cells towards the opposite surface.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments are described with reference to the following drawings, in which:

FIG. 1A shows a bioreactor module according to various embodiments;

FIG. 1B shows a bioreactor module according to various embodiments;

FIG. 1C shows a bioreactor system according to various embodiments;

FIG. 1D shows a bioreactor system according to various embodiments;

FIGS. 2A and 2B show an isometric view and a top view of a pair of bioreactor modules according to various embodiments;

FIG. 2C shows a schematic diagram of a bioreactor system according to various embodiments;

FIG. 4 shows a schematic diagram of a mechanical stimulation subsystem of the bioreactor system of FIG. 3 according to various embodiments;

FIG. 5A shows a screenshot of a computer interface for a computer controller to provide tissue mimicking electrical field induction and stimulation;

FIG. 5B shows a graph indication a comparison of experimental results for cell growth with electrical stimulation and cell growth without stimulation;

FIG. 5C shows photographs demonstrating cell growth and cell organisation with and without electrical stimulation;

FIGS. 6A-6E show experimental assessment of decellularized porcine cardiac extracellular matrix (pcECM) scaffolds' maximal cell capacity;

FIGS. 7A-7L show experimental data for compartmentalized dynamic recellularization using mono-cultures of human mesenchymal stem cells (hMSCs) and human umbilical vein endothelial cells (HUVECs);

FIGS. 8A to 8E shows experimental data for dynamic co-culturing and re-vascularization of thick pcECM scaffolds;

FIGS. 9A-9D show experimental data for in-vitro functional angiogenesis;

FIG. 10A and FIG. 10B show examples of seeding frames setup;

FIGS. 11A-11C show experimental data for screening for optimal artificial modification of pcECM adhesion sites;

FIGS. 12A-12D show experimental data for evaluating collagen binding sites and structural integrity via fluorescently labelled collagen binding peptide (CBP);

FIG. 13 shows experimental data for steady-state static culture penetration depth of hMSC on pcECM;

FIGS. 14A-14D show modelling of the pcECM support ability for HUVEC cells;

FIGS. 15A-15B shows histological stains of beating pcECM repopulated with hESC-CM and statically cultivated for 23 days; and

FIGS. 16A-16B shows neo vascularization formed during dynamic cultivation.

### DETAILED DESCRIPTION

Embodiments described below in context of the apparatus are analogously valid for the respective methods, and vice versa. Furthermore, it will be understood that the embodiments described below may be combined, for example, a part of one embodiment may be combined with a part of another embodiment.

It should be understood that the terms “on”, “over”, “top”, “bottom”, “down”, “side”, “back”, “left”, “right”, “front”, “lateral”, “side”, “up”, “down” etc., when used in the following description are used for convenience and to aid understanding of relative positions or directions, and not intended to limit the orientation of any device, or structure or any part of any device or structure.

According to various embodiments, a bioreactor module, a bioreactor system and a method for tissue cultivation may be provided. According to various embodiments, the bioreactor module, the bioreactor system and the method for tissue cultivation may be for dynamical cultivation of thick tissue slabs or engineered tissue constructs, for tissue engineering.

Accordingly, the following descriptions include set-up of a bioreactor system, set-up of a mechanical stimulation system, as well as set-up of an electrical stimulation system. All the systems may work either synchronously or separately, according to the destination of the engineered tissue and as detailed in the following description. Biological evidences of long term effects of combinatory dynamic cultivation using such a system and achieving clinically relevant constructs that are cellularized with at least two cell types which are compartmentalized in their respective niches are also provided in the description and the drawings.

According to various embodiments, there is provided a perfusion bioreactor system (in other words a bioreactor system) for cultivating tissue engineered constructs containing leaky or intact vascular-tree or partial organs containing leaky or intact vascular-tree, of human size equivalency, and a method of compartmentalized recellularization and cultivation with/without physiological mimicking electro-mechanical stimulations for the ex vivo use of biomedical professionals. The bioreactor system may include several subsystems such as a perfusion chamber (in other words a bioreactor module) with/without an imaging transparent window, electrical controllable stimulation subsystem, mechanical controllable stimulation subsystem, and a perfusion cycle. The bioreactor system with the perfusion cycle may incorporate one or more of the followings: peristaltic pumps; inlet, outlet, and low-volume circulating-cycles made out of tubing, catheters, faucets, and connectors; oxygenators, culture media reservoirs; and check valves. The bioreactor system may further enable any construct's vascular-tree (in other words a scaffold containing an inherent vascular network) to connect to at least one inlet and the collection of perfused media either through leaking drainage baths holding the construct submerged in perfused media and/or through another outlet of vein-like functionality. The scaffold containing an inherent vascular network may be a natural scaffold obtained from natural source in which the scaffold contains natural occurring vascular network. The scaffold containing an inherent vascular network may also be a synthetic scaffold made from synthetic materials in which a vascular network is pre-established during the forming of the synthetic scaffold. The ends of the vessels of the inherent vascular network may be closed such that liquid flowing into the inherent vascular network is contained within the inherent vascular network. The end of two or more vessels of the inherent vascular network may also be open such that liquid flowing into the inherent vascular network may leak from the scaffold. The bioreactor system may further conduct mechanical stimulation, through a balloon, a diaphragm, a tube or any similar device, to the construct in a physiologically mimicking manner. The bioreactor system may generate electrical stimulation with a wave form such as those corresponding to that measured in the normal tissue part in vivo, an inducing spike, an inducing pulse, a repetitive induction, or any other pattern that can be automatically computer controlled. The bioreactor system may perform tissue cultivation with perfusion, mechanical and electrical stimulation either alone or with various combinations thereof.

According to various embodiments, there is provided a method to perform compartmentalized recellularization of at least two compartments within the same construct, either individually or combined: such as the vascular tree, surface layers and construct's bulk. According to the method, seeding of the vascular network may be performed by bolus high density static perfusion of partially clamped constructs. During this static seeding, temporal rotation enabling even coating of major vascular conduits may be possible, followed by gradual dynamic perfusion at increasing shear rates over a period of a few days until physiological rates are achieved. According to the method, bulk recellularization may be enabled through either direct multi-site and multi time-points' injections or through ‘surface migratory seeding’ methodology presented firstly herein. For surface seeding maximal scaffold cell densities, within diffusion limited penetration depth, should be aspired under stabilized static culture conditions over a few days, followed by migratory diffusion induced penetration of cells towards the feeding blood vessels.

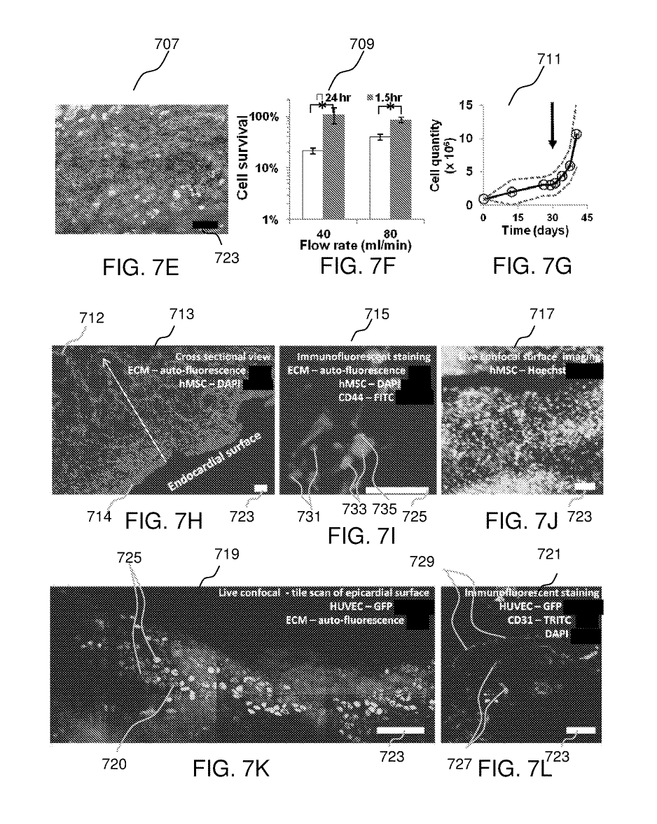

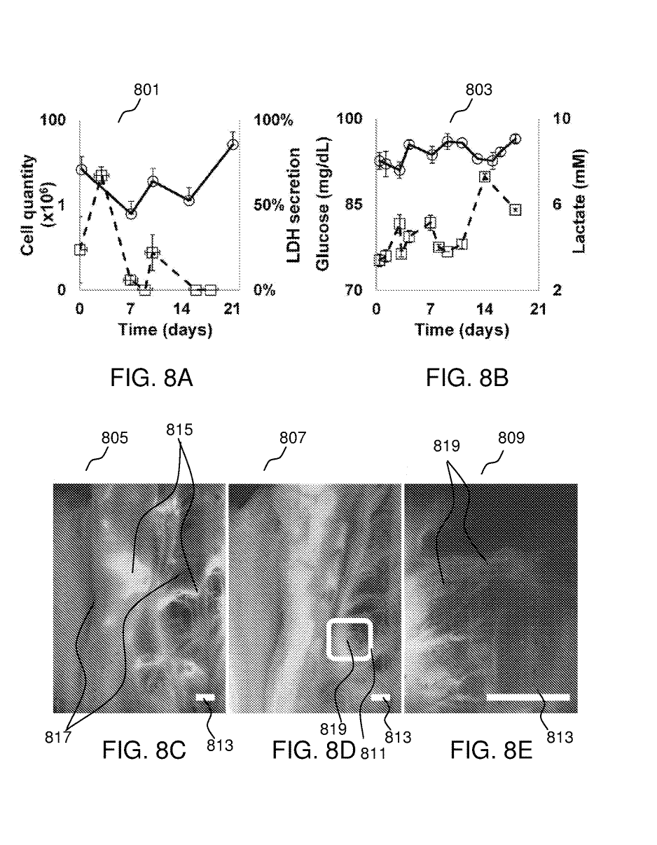

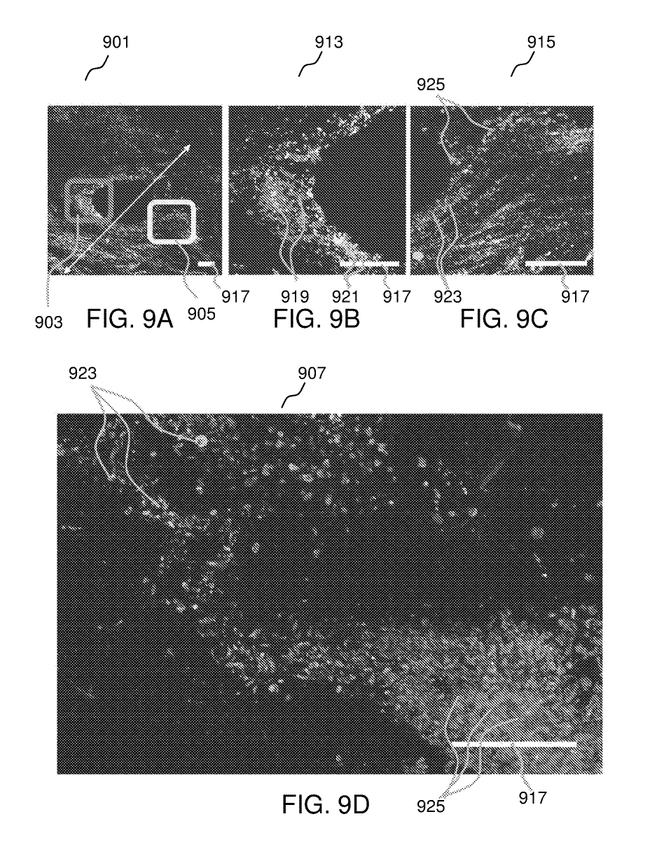

According to the method, online cell viabilities may be maintained for up to 30 days or more as shown in supporting FIGS. 7A-7L and FIGS. 8A-8E, and monitored through sterile sampling ports on the low volume (<120 ml) cycle. Online cell viability monitoring could be based on methodology correlating highly linear (R²>0.9) rates of commercial resazurin salt reduction through time to known cell densities (cell/volume) and quantities when multiplied by the total dead volume of the low volume cycle (FIGS. 8A-8E). Alternative measurements may include the indirect measurements of biochemical representatives of cell metabolism (such as glucose consumption, lacatate production, osmolarity, production of —NH₄⁺ ions etc., FIG. 8A-8E) or the monitoring through histology and microscopy of cells expressing coloured fluorescent proteins, for example a green fluorescent protein indicated by 815 and a red fluorescent protein indicated by 817 in FIG. 8C, when viable to study their morphology and organization through microscopy within the tissue (FIGS. 8A-8E and 9A-9E).

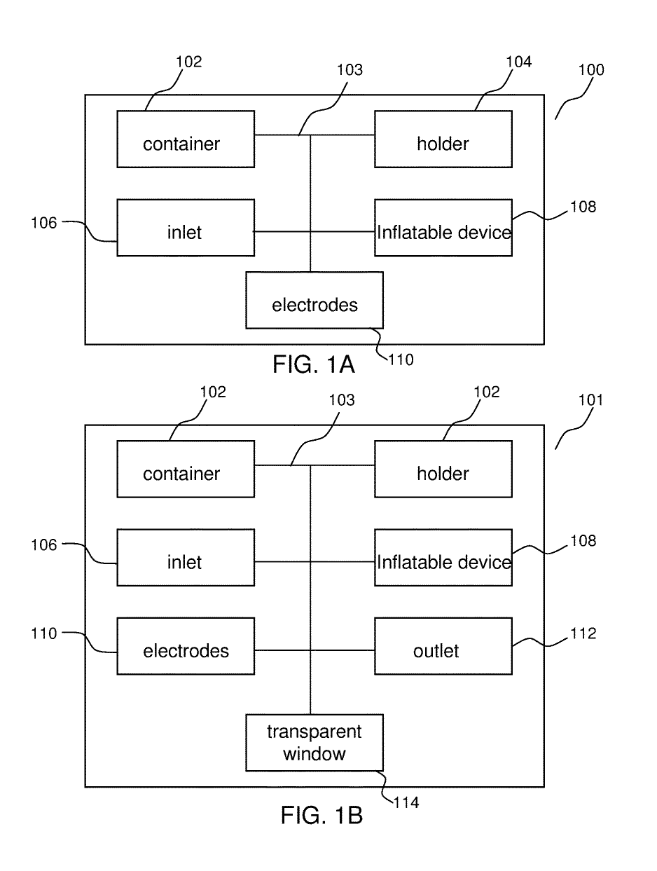

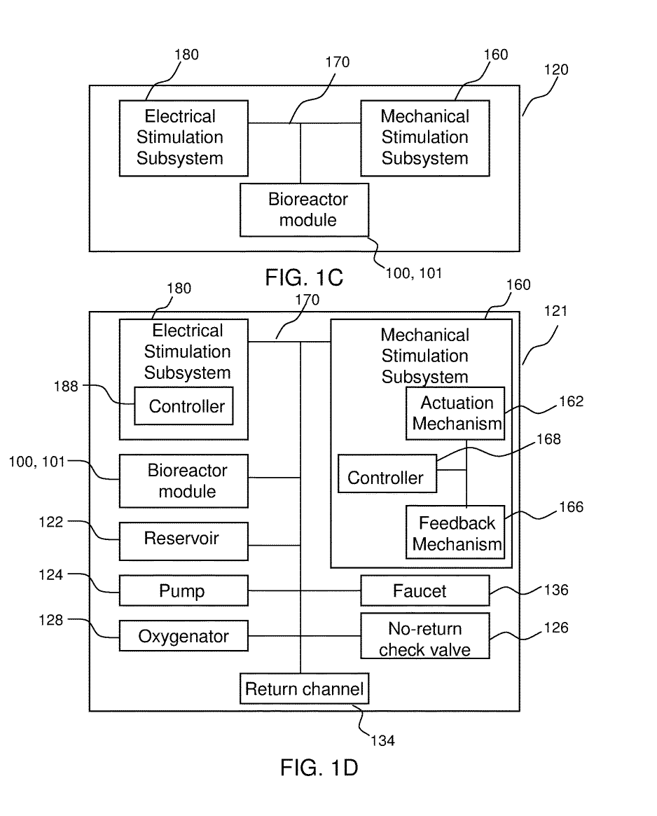

FIG. 1A shows a bioreactor module 100 according to various embodiments. The bioreactor module 100 may include a container 102. The bioreactor module 100 may further include a holder 104 removably receivable in the container 102. The holder 104 may be adapted to hold a scaffold containing an inherent vascular network. The bioreactor module 100 may further include an inlet 106 connectable to a vessel of the inherent vascular network of the scaffold. The bioreactor module 100 may further include an inflatable device 108 disposed within the container. The inflatable device 108 may have a conduit extending through the wall of the container 102. The bioreactor module 100 may further include a pair of electrodes 110 attached to opposing walls of the container 102. The container 102, the holder 104, the inlet 106, the inflatable device 108 and the pair of electrodes 110 may be connected with each other directly or indirectly, like indicated by lines 103.

In other words, the bioreactor module 100 may include a containment element such as a basin, a tank, a bath, a tub or any other element suitable for containing fluid.

In other words, the bioreactor module 100 may further include an attachment element such as a clamping element, a gripping element, a fastening element, a coupling element, or other element suitable for attaching a scaffold to the attachment element. The attachment element may receive the scaffold such that when the attachment element, with the scaffold received in the attachment element, is introduced into the containment element, the scaffold may be contained within an enclosed space of the containment element. The scaffold may include a structure capable of supporting tissue formation. The scaffold may be made of natural material, synthetic material, or biodegradable material etc. The scaffold may include extracellular matrix (ECM) of a tissue. The scaffold may further include decellularized ECM of a tissue. The scaffold may contain a vascular network. The scaffold may contain naturally inherent occurring vascular network for a scaffold obtain from a natural source. For example, the scaffold may contain blood vessel network or fluidic network. The scaffold may also contain a pre-established vascular network in a synthetic scaffold. In other words, the vascular network is formed in the synthetic scaffold. The attachment element may be capable of being introduced into the containment element and may also be capable of being removed from the containment element.

In other words, the bioreactor module 100 may further include an input element which may be capable of being directly connected to one of the vessel of the vascular network of the scaffold. The input element may be configured for flowing fluid through the input element into the vessel of the vascular network of the scaffold. The input element may be in the form of a tube, a catheter, a fluid connector, etc. The input element may be extended through an opening of the containment element and into the enclosed space of the containment element for connecting with the vessel of the vascular network of the scaffold held within the enclosed space of the containment element by the attachment element inserted into the containment element.

In other words, the bioreactor may further include an expandable element attached to the containment element. The expandable element may be located within the enclosed space of the containment element and connected to at least one wall of the containment element. According to various embodiments, the expandable element may be located near a bottom of the containment element or near a top of the containment element. The expandable element may be expanded and contracted such that the expansion and contraction of the expandable element may cause a mechanical stimulation to the scaffold held in the containment element. The expandable element may be made of elastic material and may be in the form of a balloon, or a tube such that air, gas or liquid may be pumped into the expandable element to cause the expansion. The expandable element may then be contracted from the expanded state to return to the original state by releasing the air, gas or liquid previously pumped into the expandable element.

In other words, the bioreactor module 100 may further include a pair of electrical conductors within the enclosed space of the containment element and attached to opposing walls of the containment element. The pair of electrical conductors may be spaced apart and may be capable of generating an electrical pulse or signal through a fluid contained in the containment element. The pair of electrical conductors may be adapted to generate electrical pulses that mimick the electrical pulses of the desired tissue to be cultivated from the scaffold.

FIG. 1B shows a bioreactor module 101 according to various embodiments. The bioreactor module 101 may, similar to the bioreactor module 100 of FIG. 1A, include a container 102. The bioreactor module 101 may, similar to the bioreactor module 100 of FIG. 1A, further include a holder 104 removably receivable in the container 102. The holder 104 may be adapted to hold a scaffold containing an inherent vascular network. The bioreactor module 101 may, similar to the bioreactor module 100 of FIG. 1A, further include an inflatable device 108 disposed within the container. The inflatable device 108 may have a conduit extending through a wall of the container 102. The bioreactor module 101 may, similar to the bioreactor module 100 of FIG. 1A, further include a pair of electrodes 110 attached to opposing walls of the container 102. The bioreactor module 101 may further include an outlet 112 in a wall of the container 102. The bioreactor module 101 may further include a transparent window 114 covering an opening of the container 102. The container 102, the holder 104, the inlet 106, the inflatable device 108, the pair of electrodes 110, the outlet 112 and the transparent window 114 may be connected with each other directly or indirectly, like indicated by lines 103.

According to various embodiments, the inflatable device 108 may include a balloon, an elastic tube (e.g., medical latex) or a diaphragm.

According to various embodiments, the bioreactor module 101 may include a pair of holders 104. According to various embodiments, the container 102 may be adapted to receive the pair of holders 104 so that the pair of holders 104 may be separated by a distance from each other, wherein the distance is variable.

According to various embodiments the scaffold may include a natural scaffold containing a natural inherent vascular network. According to various embodiments, the scaffold may include a synthetic scaffold containing an inherent vascular network formed in the synthetic scaffold. According to various embodiments, an end of two or more vessels of the inherent vascular network of the scaffold may be opened.

FIG. 1C shows a bioreactor system 120 according to various embodiments. The bioreactor system 120 may include a bioreactor module 100, 101 as described above. The bioreactor system 120 may further include a mechanical stimulation subsystem 160 adapted to control the inflatable device 108 of the bioreactor module 100, 101 to generated mechanical stimulation by controlling the inflation of the inflatable device 108. The bioreactor system 120 may further include an electrical stimulation subsystem adapted to control the pair of electrodes 110 of the bioreactor module 100, 101 to generate electrical pulses from the pair of electrodes. The mechanical stimulation subsystem 160, the electrical stimulation subsystem 180, and the bioreactor module 100 may be connected with each other directly or indirectly, like indicated by lines 170.

In other words, the bioreactor system 120 may include three inter-related subsystems or circuits. The subsystems may include a fluid flow cycle subsystem or a perfusion cycle subsystem which may generate a fluid flow in a cycle from a fluid reservoir to the bioreactor module 100 or 101 and back to the fluid reservoir. Fluid may flow from the fluid reservoir through conduits to the input element of the bioreactor module 100 or 101 as described above. The fluid may then flow through the vascular network of the scaffold held in the containment element of the bioreactor module 100 or 101. Fluid may leak from the vascular network of the scaffold to fill the containment element of the bioreactor module 100 or 101. The fluid may then flow out of the containment element via an outlet in the containment element of the bioreactor module 100 or 101. Conduits may be connected to the outlet to flow the fluid back to the fluid reservoir of the bioreactor module 101. A pump may be used to generate the flow from the fluid reservoir to the bioreactor module 100 or 101 and back to the fluid reservoir. The subsystems may further include a mechanical stimulation subsystem which may control the expansion and contraction of the expandable element in the bioreactor module 100 or 101. The mechanical stimulation subsystem may include a controller connected to an actuator for controlling the amount of air, gas or liquid being pumped into or out of the expandable element. The mechanical stimulation subsystem may be a closed loop system which may include feedback mechanism to monitor the expansion and contraction of the expandable element. The subsystems may further include an electrical stimulation subsystem which may include an external electrical circuit connected to the pair of spaced apart conductors of the bioreactor module 100 or 101 such that when fluid fills the containment element such that portions of the pair of spaced apart conductors may be immersed in the fluid, the electrical circuit may be considered closed and electrical signals or pulses may be generated within the closed loop electrical circuit. The electrical signals or pulses may be transmitted by one of the pair of conductors through the fluid to the other of the pair of conductors.

FIG. 1D shows a bioreactor system 121 according to various embodiments. The bioreactor system 121 may, similar to the bioreactor system 120 of FIG. 1C, include a bioreactor module 100, 101 as described above. The bioreactor system 121 may, similar to the bioreactor system 120 of FIG. 1C, further include a mechanical stimulation subsystem 160 adapted to control the inflatable device 108 of the bioreactor module 100, 101 to generated mechanical stimulation by controlling the inflation of the inflatable device 108. The bioreactor system 121 may, similar to the bioreactor system 120 of FIG. 1C, further include an electrical stimulation subsystem 180 adapted to control the pair of electrodes 110 of the bioreactor module 100, 101 to generate electrical pulses from the pair of electrodes. The mechanical stimulation subsystem 160, the electrical stimulation subsystem 180, and the bioreactor module 100 may be connected with each other directly or indirectly, like indicated by lines 170.

According to various embodiments, the electrical pulses may include custom designed electrical pulses.

According to various embodiments, the mechanical stimulation subsystem 160 may include a controller 168. The mechanical stimulation subsystem 160 may further include an actuation mechanism 162 adapted to inflate the inflatable device 108 based on instructions received from the controller 168. The mechanical stimulation subsystem may further include a feedback mechanism 166 adapted to measure the pressure of the inflatable device 108.

According to various embodiments, the actuation mechanism 162 may include an actuator and a hydraulic pump adapted to supply pressurized fluid to the inflatable device 108.

According to various embodiments, the actuation mechanism 162 may include an actuator and a pneumatic pump adapted to supply pressurized air to the inflatable device 108.

According to various embodiments, the actuation mechanism 162 may include an actuator and a pneumatic pump adapted to supply pressurized gas to the inflatable device 108.

According to various embodiments, the feedback mechanism 166 may include a pressure transducer.

According to various embodiments, the electrical stimulation subsystem 180 may include a controller 188 adapted to send electrical signals to the pair of electrodes 110 of the bioreactor module 100, 101 to generate the electrical pulses.

According to various embodiments, the electrical pulses may include tissue mimicking electrical wave form.

According to various embodiments, the bioreactor system 121 may further include a reservoir 122 adapted to contain culture medium.

According to various embodiments, the bioreactor system 121 may include a pump 124 adapted to pump culture medium from the reservoir 122 to the bioreactor module 121.

According to various embodiments, the bioreactor system 121 may further include an oxygenator 128 located along a fluid communication between the pump 124 and the bioreactor module 100, 101 to maintain a predetermined oxygen level in the culture medium.

According to various embodiments, the bioreactor system 121 may further include a no-return check valve 126 located along a fluid communication between the pump 124 and the bioreactor module 100, 101.

According to various embodiments, the bioreactor module 100, 101 may be located in an incubator. The incubator may be a standard carbon dioxide (CO₂) incubator. The incubator may be maintained at a predetermined temperature. The predetermined temperature may be 37° C.

According to various embodiments, the bioreactor system 121 may further include a faucet 136 located along a fluid communication from the bioreactor module. The faucet 136 may be a three-way faucet. A sampling port may be connected to the faucet 136. Samples of the culture medium may be taken from the sampling port. Concentration-dependent measurements may be taken to assess cell quantity and metabolic state.

According to various embodiments, the bioreactor system 121 may further include a return channel 134 adapted to return culture medium from the bioreactor module 100, 101 to the reservoir 122.

According to various embodiments, an in-vitro method for tissue cultivation may include connecting a vessel of an inherent vascular network of a scaffold to an inlet 106 of a bioreactor module 100, 101 and perfusing the scaffold via the inlet 106 of the bioreactor module 100, 101.

According to various embodiments, an in-vitro method for tissue cultivation may include seeding an interior of a vessel of an inherent vascular network of a scaffold with a first cell type, seeding an exterior surface of the scaffold with a second cell type, and perfusing the inherent vascular network of the scaffold with culture medium to facilitate compartmentalized co-cultivation of the first cell type and the second cell type in different niches of the tissue.

According to various embodiments, perfusing the scaffold via the inlet of the bioreactor with culture medium may include perfusing initially with a first culture medium; and replacing the first culture medium with a second culture medium gradually to ensure cell acclimation to the culture media change towards co-culture conditions.

According to various embodiments, the first culture medium may include M199 medium or Endothelial Growth Medium-2 medium.

According to various embodiments, the second culture medium may include culture medium for supporting the co-culture conditions.

According to various embodiments, the second culture medium comprises alpha modified Eagle's medium or Endothelial Growth Medium-2 or mTEASER or Roswell Park Memorial Institute medium.

According to various embodiments, the in-vitro method may further include adding growth factors and cytokines such as human recombinant vascular endothelial growth factor (VEGF) basic fibroblast growth factor (bFGF) or any other factor (cell type dependent) to the culture medium which diffusion can cause cell survival, proliferation, polarization, migration and integration.

According to various embodiments, the seeding of the interior of the vessel of an inherent vascular network of a scaffold may include rotating the scaffold to coat the vessel with the first cell type.

According to various embodiments, the seeding of the exterior surface of the scaffold may include seeding by injection into the exterior surface of the scaffold.

According to various embodiments, the seeding of the exterior surface of the scaffold may include seeding by pipettation on the exterior surface of the scaffold.

According to various embodiments, the first cell type may include endothelial cells and the second cell type may include pericytic cells.

According to various embodiments, the first cell type may include endothelial cells and the second cell type may include parenchymal cells.

According to various embodiments, the scaffold may include decelluralized extracellular matrix with an inherent vascular network preserved.

According to various embodiments, the method for tissue cultivation may further include connecting the vessel of the inherent vascular network of the scaffold to an inlet of a bioreactor module as described herein, wherein perfusing through the inherent vascular network of the scaffold may include perfusing via the inlet of the bioreactor module.

According to various embodiments, an in-vitro method for tissue cultivation may include seeding a surface of a scaffold and perfusing the scaffold from an opposite surface of the scaffold through the scaffold and towards the seeded surface with culture medium to provide flow of nutrients and oxygen through to create a nutrient/oxygen gradient between the opposite surface and the seeded surface of the scaffold to cause migratory diffusion induced penetration of cells towards the opposite surface.

According to various embodiments the scaffold may include a scaffold containing an inherent vascular network.

According to various embodiments, the scaffold may include decelluralized extracellular matrix with an inherent vascular network preserved.

According to various embodiments, the method for tissue cultivation may further include connecting the vessel of the inherent vascular network of the scaffold to an inlet of a bioreactor module as described herein, wherein perfusing the scaffold comprises perfusing via the inlet of the bioreactor module through the inherent vascular network. The flow of nutrients and oxygen through the inherent vascular network may create a nutrient/oxygen gradient between the inherent vascular network and the seeded surface of the scaffold to cause migratory diffusion induced penetration of cells towards the inherent vascular network.

According to various embodiments, the scaffold may include decelluralized extracellular matrix.

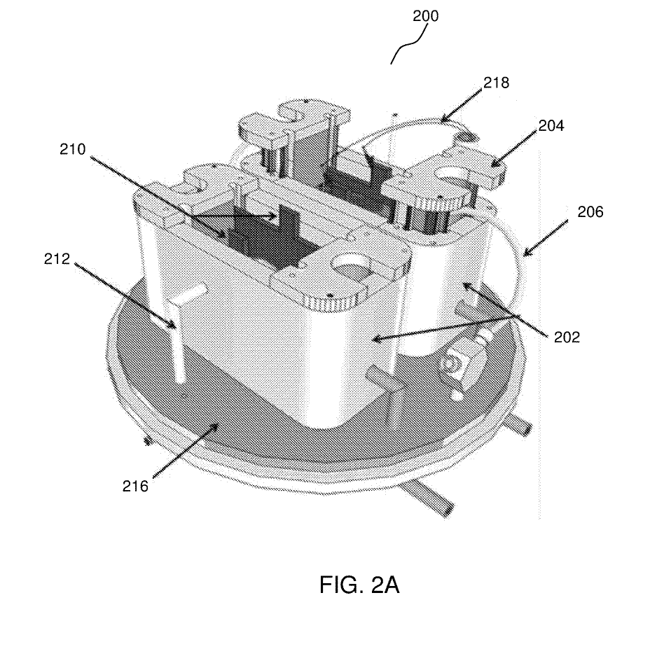

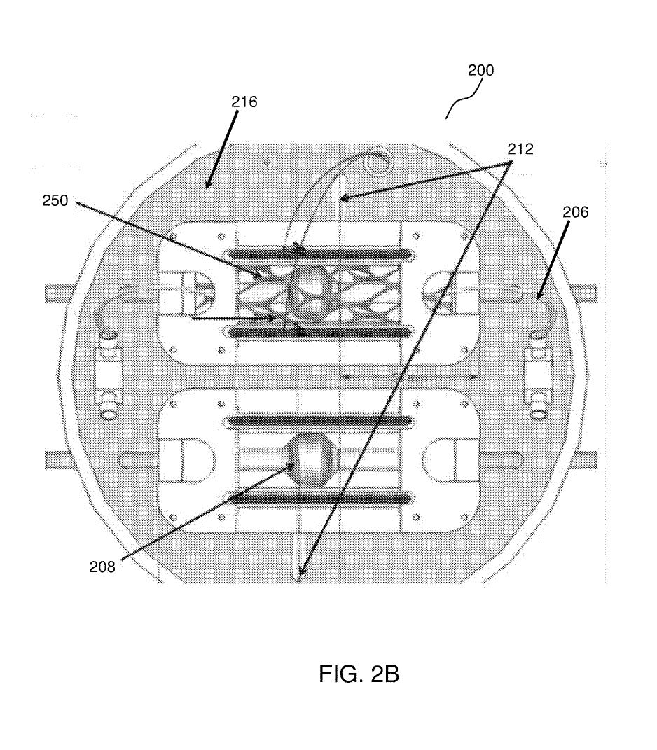

FIGS. 2A and 2B show an isometric view and a top view respectively of a pair of bioreactor modules according to various embodiments. As shown, the bioreactor module 200 may include a container 202. According to various embodiments, the container 202 may have different dimensions depending on the sizes of the tissue to be cultivated. The various dimensions of the container 202 may range from a dimension suitable for cultivating small tissue segment to a dimension suitable for cultivating an entire organ. The container 202 may include an opening in a top surface of the container 202. In other words, the container 202 may include a closed base, side wall(s) and an opened top. The container 202 is shown to be a cuboid. In various embodiments, the container 202 may be cylindrical, hexagonal prism or any other suitable shapes.

The bioreactor module 200 may further include a holder 204. The holder 204 may be adapted such that it is removably receivable in the container 202. Accordingly, the holder 204 may be inserted into the container 202 via the opening in the top surface of the container 202, and the holder 204 may be removed from the container 202 from the top surface of the container 202. In an implementation, the container 202 may include slots or grooves along the side wall(s), and the holder 204 may include corresponding protruding members slidably receivable in the slots or grooves for sliding the holder 204 into or out of the container 202.

The holder 204 may further be adapted to hold a scaffold. The scaffold may contain an inherent vascular network. In various implementations, the holder 204 may include a clamping mechanism for clamping the scaffold, a gripping mechanism for gripping the scaffold, a hook for hooking the scaffold, or an attachment mechanism for attaching the scaffold to the holder 204. In use, the scaffold may be fitted onto the holder 204 when the holder 204 is out of the container 202. After which, the holder 204 together with the scaffold may be inserted into the container 202. FIG. 2B illustrates an example of the holder 204 holding the scaffold 250 when inserted in the container 202. The holder 204 may be configured to accommodate scaffold of different sizes. According to various embodiments, the holder 204 may have different dimensions depending on the sizes of the scaffold to be accommodated in the container 202. According to various embodiments, the bioreactor module 200 may include a pair of holders 204. The pair of holders 204 may also be inserted into the container 202 in a spaced apart configuration. The pair of holders 204 may accommodate scaffold of different sizes by being configured to vary a distance between the pair of holders 204 in the spaced apart configuration when inserted into the container 202. In other words, the container 202 may be adapted to receive the pair of holders 204 so that the pair of holders 204 may be separated by a distance from each other, wherein the distance may be variable.

The bioreactor module 200 may further include an inlet 206. The inlet 206 may be in the form of a tube or a catheter. The inlet 206 may be adapted to be connectable to a vessel of the inherent vascular network of the scaffold. In this manner, culture medium may flow from the inlet 206 into the vessel of the inherent vascular network of the scaffold to provide perfusion stimulation. In an implementation, the inlet 206 in the form of a tube may be connected to one of the vessel of the inherent vascular network of the scaffold by inserting the tube into the vessel. In another implementation, the scaffold containing inherent vascular network may be pre-prepared with a catheter sutured in place and the inlet 206 may be directly connected to the catheter.

The bioreactor module 200 may further include an inflatable device 208 disposed substantially near a base of the container 202. The inflatable device 208 may be a balloon, a diaphragm or any similar device. The inflatable device 208 may include a conduit extending from the inflatable device 208 through a wall of the container 202 and out of the container 202. The conduit may allow fluid, air or gas to flow into the inflatable device 208 to inflate the device. The conduit may be connected to external apparatus for generating the flow of fluid, air or gas through the conduit into the inflatable device 208 for providing mechanical stimulation to a scaffold when the scaffold is held in the container 202.

The bioreactor module 200 may further include a pair of electrodes 210 attached to opposing walls of the container 202. The pair of electrodes 201 may be connected to external apparatus for generating electrical signals via electrical wires 218. The external apparatus may provide electrical signals to the pair of electrodes for generating electric pulses to provide electrical stimulation to a scaffold when a scaffold is held in the container 202.

Advantageously, the bioreactor module 200 may be easy to use and operate. A user may easily fit a scaffold 250 onto the holder 204 when the holder 204 is outside the container 202 such that the scaffold 250 is being held by the holder 204. After which, the user may easily insert the scaffold 250 into the container 202 of the bioreactor 200 by inserting the holder 204 into the container 202 of the bioreactor 200. Alternatively, the scaffold 250 may be easily placed within the container 202 of the bioreactor and secured into place by the pair of holders 204. The user may then connect the inlet 206 to the scaffold 250. As the mechanical stimulation element, such as the inflatable device 208, and the electrical stimulation element, such as the pair of electrodes 210, are already in the container 202 of the bioreactor module 200, the user do not need to perform additional steps of attaching mechanical stimulation element or electrical stimulation element to the scaffold. In either case, all operation can be performed under aseptic conditions to ensure scaffold sterility, and the minimal handling required may potentially reduce the risk of contaminating the scaffold as the user could minimize contact with the scaffold.

The scaffold 250 may be a natural scaffold containing a natural inherent vascular network. The scaffold 250 may also be a synthetic scaffold containing an inherent vascular network formed inside the synthetic scaffold. According to various embodiments, the ends of the vessels of the inherent vascular network of the scaffold 250 may be closed such that liquid flowing through the inherent vascular network of the scaffold 250 may be kept within the inherent vascular network of the scaffold 250. According to various embodiments, an end of two or more vessels of the inherent vascular network of the scaffold 250 may be opened such that liquid flowing through the inherent vascular network of the scaffold 250 may leak from the scaffold 250. In other words, the bioreactor module 200 may be used to support tissue cultivation of a leaky scaffold. A leaky scaffold may be a scaffold with ends of the inherent vascular network opened. Liquid may be flowed via the inlet 206 of the bioreactor module 200 into the inherent vascular network of the leaky scaffold. Liquid may then leak from the opened ends of the vessels of the inherent vascular network of the leaky scaffold. The leakage from the scaffold may be contained within the container 202 of the bioreactor module 200. An outlet 212 may be provided in the container 202 for draining the liquid collected in the container 202. Thus, the bioreactor module 200 may support perfusion of leaky scaffold or leaky construct.

Advantageously, the bioreactor module 200 may be suitable for anything between an entire organ and a small simple tissue segment. It may be made possible by having different holders and distances to accommodate different scaffold or constructs all sharing common criteria: the perfusion through inherent (natural) or pre-established (for synthetic materials) vascular-like network; and the vascular like network does not have to be complete or closed. The bioreactor module 200 may be designed to support ‘leaky’ constructs with open circulation which may be an advantage over conventional bioreactor setups for whole-organ perfusion.

As shown in FIG. 2A, the bioreactor module 200 may include two sets of the containers 202, each having the holder 204, the inlet 206, the inflatable device 208 and the pair of electrodes 210. According to various embodiments, the bioreactor module 200 may include different number of sets of the containers 202 having the holder 204, the inlet 206, the inflatable device 208 and the pair of electrodes 210.

According to various embodiments, the bioreactor module 200 may further include a base plate 216 for supporting the container 202. When the bioreactor module 200 includes multiple sets of the containers 202, the multiple sets of the containers 202 may be supported by a single or multiply segmented base plate 216.

According to various embodiments, the bioreactor module 200 may further include an outlet 212 extending out of the container 202. The outlet 212 may be adapted to drain culture medium flowing into the container 202 via the inlet 206 through the vessel in the inherent vascular network of the scaffold.

According to various embodiments, the bioreactor module 200 may further include a housing 340 enclosing the bioreactor module 200. The housing 340 may be a transparent housing or may be a housing with a transparent window at the top surface of the housing 340. The transparent housing or the transparent window may allow imaging of the scaffold during various stages of tissue cultivation for data collections and analysis. The transparent housing or the transparent window may enable online monitoring and imaging under sterile culture conditions.

FIG. 3 shows a schematic diagram of a bioreactor system according to various embodiments. The bioreactor system 300 may include a bioreactor module 200. The bioreactor module 200 may be fluidly connected to a culture medium reservoir 322, a pump 324, a no-return check valve 326, an oxygenator 328, faucets 336, and a return channel 334. According to various embodiments, the reservoir 322 may contain culture medium for circulation. The pump 324 may be arranged to be in direct fluid connection with the reservoir 322 such that the pump 324 may draw culture medium from the reservoir 322 and pump culture medium from the reservoir 322 to the bioreactor module 200. In other words, the pump 324 provides the actuation to circulate the culture medium. The no return check valve 326 may be arranged to be located along a fluid communication after the pump 324 to prevent back flow of the culture medium back into the pump 324. The oxygenator 328 may be arranged to be located along a fluid communication after the no return check valve 326 so as to maintain a predetermined level of oxygen levels in the culture medium before the culture medium flows into the bioreactor module 200. Accordingly, the bioreactor module 200 may be arranged to be located along a fluid communication after the oxygenator 328. The return channel 334 may fluidly connect the bioreactor module 200 back to the reservoir 322 through the pump 324. The return channel 334 may provide a fluid communication for culture medium to be pumped back by pump 324 to the reservoir 322. The bioreactor system 300 may further include a bypass channel 332 to provide a fluid communication for the culture medium to bypass the reservoir. The faucets 336 may be located anywhere along the bypass channel 332 or at an end of the bypass channel 332. Sampling port may be connected to each of the faucets 336. Samples of the culture medium may be taken from the sampling port. Concentration-dependent measurements may be taken to assess cell quantity and metabolic state.

According to various embodiments, the bioreactor system 300 may include a housing 340 enclosing the bioreactor module 200 as shown in FIG. 3. The housing may be a glass casing configured to contain the bioreactor module 200 such that the bioreactor module 200 may be kept in a sterile environment within the housing 340.

According to various embodiments, the bioreactor system 300 may further include a mechanical stimulation subsystem adapted to control the inflatable device 208 of the bioreactor module 200 to generate mechanical stimulation by controlling the inflation of the inflatable device 208. The bioreactor system 300 may further include an electrical stimulation subsystem adapted to control the pair of electrodes 210 of the bioreactor module 200 to generate electrical stimulation by transmitting electric pulses from the pair of electrodes 210.

As shown in FIG. 3, the bioreactor system 300 may include a medium reservoir 322, which may supply culture media through a peristaltic pump 324, a check valve 326 and an oxygenator 328 to the perfusion module 200 within the housing 340. Two separate lines 332, 334 may be responsible for drainage either back to the reservoir (dashed line 334) or using a smaller volume cycle (reservoir bypass) for cell quantifications (dotted line 332). In FIG. 2C, the gray shade represents a standard CO₂ incubator 342. Three-way faucets 336 and sampling ports may be located on the two drainage lines 332, 334. The bioreactor module 200 may have two identical containers 202 for statistical repetition purposes. As shown in FIG. 2A and 2B, the bioreactor module 200 may be drained from the side of the container 202 using the low volume cycle (in other words through an outlet 212). A thick pcECM matrix 250 (in other words a scaffold containing an inherent vascular network), as represented by a mesh in FIG. 2B, is fed by a 24 gauge silicon catheter (in other words an inlet) 206 and is held in place by two holders 204. The bioreactor module 200 may further include a balloon (in other words an inflating device) 208 and a pair of electrodes 210 which may enable mechanical and electrical stimulation.

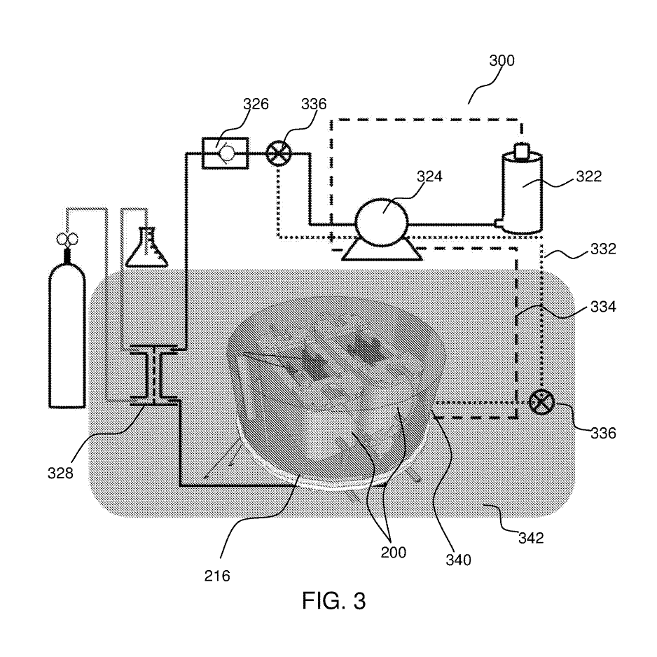

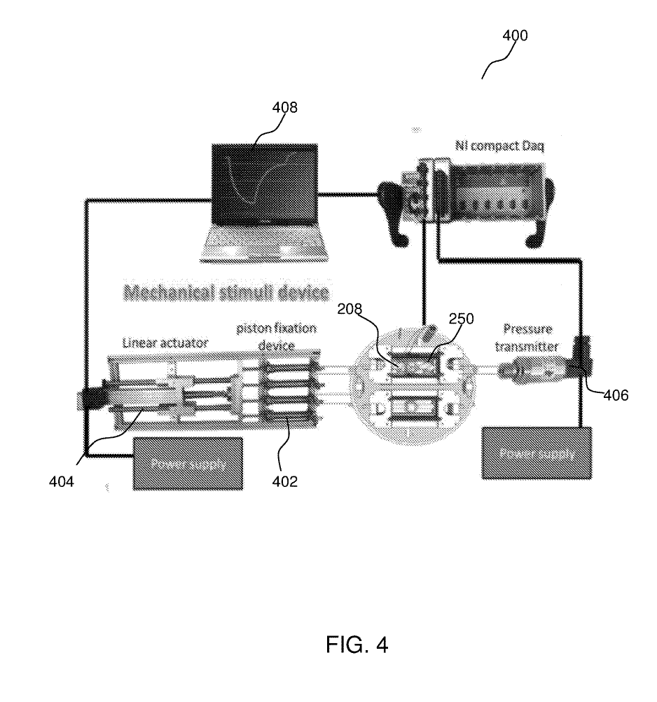

FIG. 4 shows a schematic diagram of a mechanical stimulation subsystem 400 of the bioreactor system 300 according to various embodiments. The mechanical stimulation subsystem 400 may include a controller 408. The controller 408 may be in the form of a computer device or any other processing devices. An actuation mechanism may be connected to the controller 408. The actuation mechanism may be adapted to inflate the inflatable device 208 of the bioreactor module 200 by pressurising the inflatable device 208 based on instructions received from the controller 408. According to various embodiments, the actuation mechanism may include a hydraulic pump 402 and an actuator 404. The hydraulic pump 402 may include piston and the actuator 404 may include linear actuator. The hydraulic pump 402 and the actuator 404 may be adapted to supply pressurized fluid to the inflatable device 208. According to various embodiments, the actuation mechanism may include an actuator and a pneumatic pump adapted to supply pressurized air to the inflatable device 208. According to various embodiments, the actuation mechanism may include an actuator and a pneumatic pump adapted to supply pressurized gas to the inflatable device 208.

According to various embodiments, the mechanical stimulation subsystem 400 may further include a feedback mechanism 406. The feedback mechanism may be adapted to measure the pressure of the inflatable device 208. According to various embodiments, the feedback mechanism may be a pressure transducer.

As shown in FIG. 4, the mechanical stimulation subsystem 400 may include positive pressure stainless steel (food grade) pistons 402 and a computer controlled motor 404 that pumps hydraulic or pneumatic means into a latex based balloon catheter or tubing (in other words the inflating device) 208 located within the perfusion bath (in other words the bioreactor module) 200 underneath the held matrix (in other words the scaffold) 250. At the other side the exit hole is connected to a pressure transducer (in other words a feedback mechanism) 406 for measurement of pressures and forces at play and monitoring of correct function according to desired stimulation program by the computer (in other words a controller) 408.

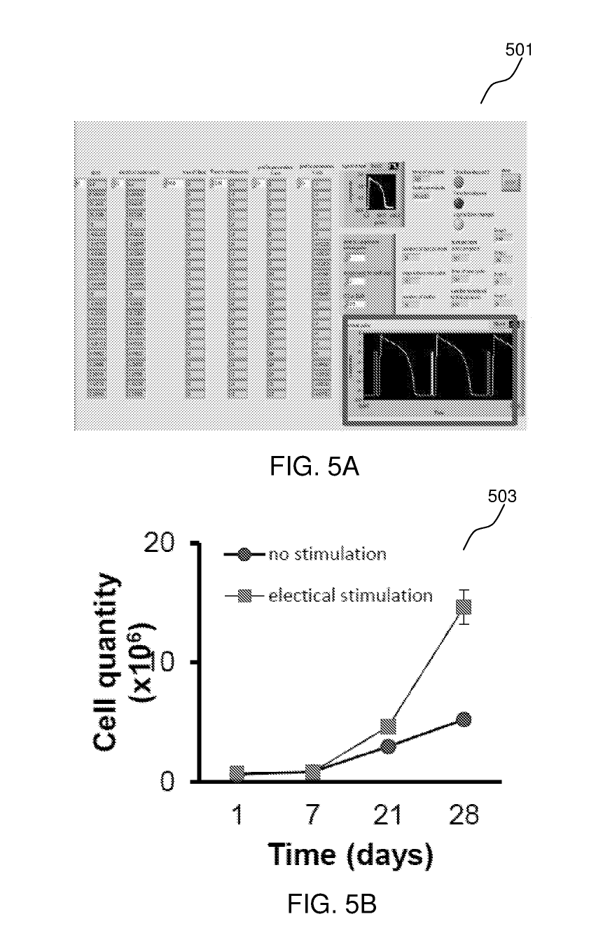

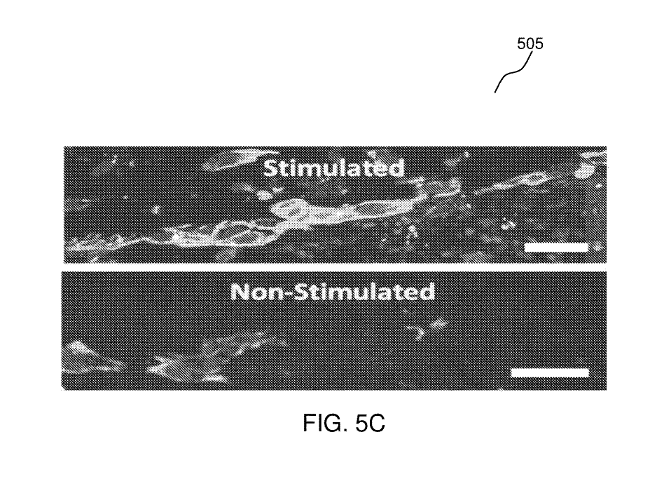

According to various embodiments, the electrical stimulation subsystem may include a controller adapted to send electrical signals to the pair of electrodes 210 of the bioreactor module 200 to generate electric pulses for electrical stimulation. The controller may be in the form of a computer device or any other processing devices. FIG. 5A shows a screenshot 501 of a computer interface for a computer controller to provide tissue mimicking electrical field induction and stimulation. The custom designed software may be used to tailor the various electrical signals and frequencies (including tissue mimicking wave form, spike form, pulse or pattern or any other direct/alternating forms of stimulation). In other words, the computer controller may send electrical signals to the pair of electrodes 210 to generate pulses that mimic the actual electrical pulses as measured within the relevant tissue slab, i.e. the computer controller may custom designed electrical signals to be sent to the pair of electrodes 210 to generate custom designed electrical pulses. The computer controller may also send electrical signals to the pair of electrodes 210 to generate pulses to stimulate the cells to generate their own electrical signal. FIG. 5B shows a graph 503 indicating a comparison of experimental results for cell growth with electrical stimulation and cell growth without stimulation. FIG. 5C shows photographs 505 demonstrating cell morphology and organization with and without electrical stimulation.

According to various embodiments, the bioreactor systems 300 and the bioreactor modules 200 may be used to cultivate tissue using scaffold containing inherent vascular network. The use of scaffolds which contain inherent vascular network templates may be advantageous as those scaffolds could be subsequently cellularized with vascular cells, and instantly utilized to support the growing tissue.

Recent advances in tissue decellularization techniques have produced natural extracellular matrix (ECM) out of various tissues and organs. ECM scaffolds may serve as a template for recellularization of small tissue constructs or even whole organs. Accordingly, cultivation of substitute tissue mimetic using ECM scaffolds may be possible in a lab condition. To demonstrate the use of the bioreactor system 300, and the bioreactor module 200 as described herein, experimental data and results are provided in the following for cultivation using a decellularized porcine cardiac extracellular matrix (pcECM).

Isolated large (3×7×1 cm) ventricular pcECM slabs preserving leaky vascular networks may be used. The pcECM major advantages were hypothesized to be in maintaining perfusable, even if not intact, supportive vascular network of physiological relevant dimensions on the one hand while on the other hand, being of clinically feasible recellularizing sizes compared to whole-organ templates. This biomaterial is used herein as an example for the applicability of the bioreactor system using one of the most complicated soft-tissues—the heart—for the engineering of tissue constructs with human relevant physiological dimensions and functionalities. Results have shown that this bioreactor system may thus be used to either produce mature constructs for implantation or for the ex vivo cultivation of platforms serving as human mimetic tissues for drug screening purposes.

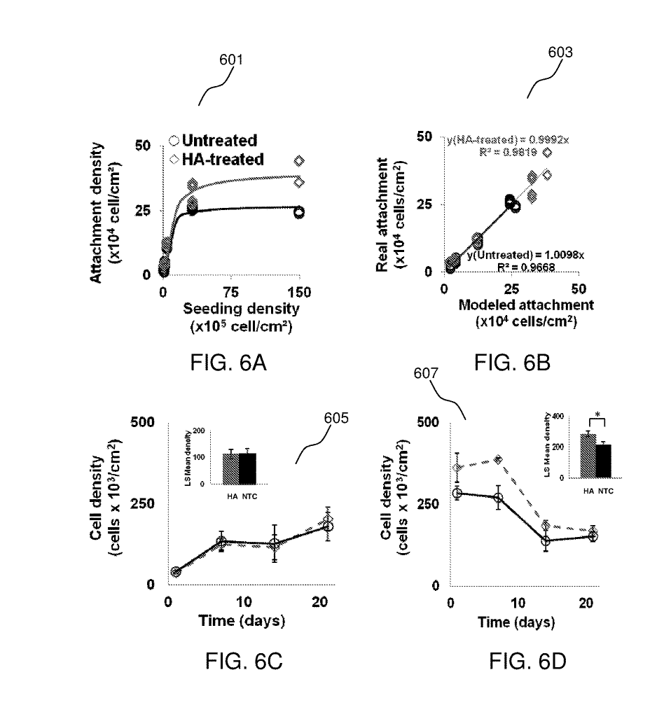

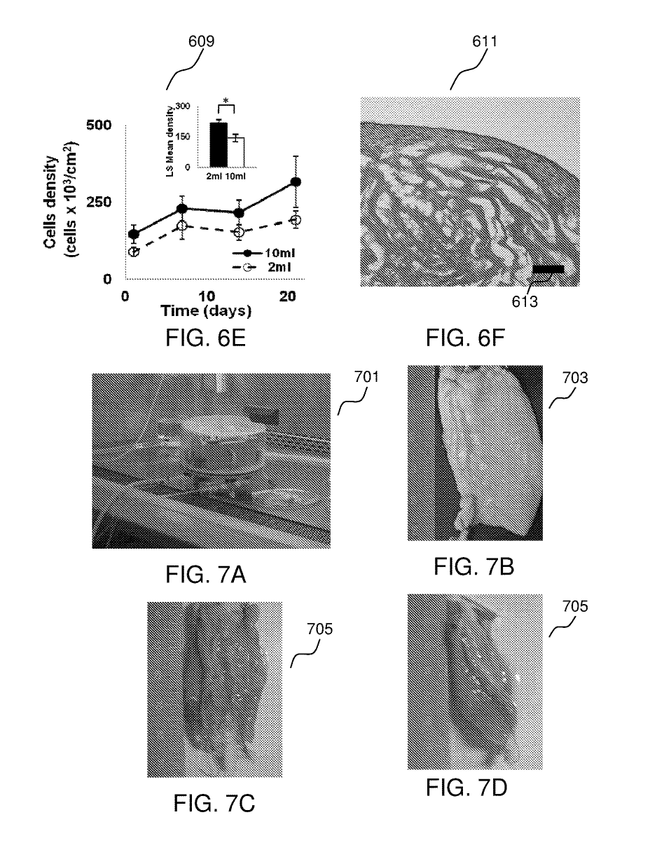

FIGS. 6A-6E show experimental assessment of decellularized porcine cardiac extracellular matrix (pcECM) scaffolds' maximal cell capacity under static (i.e. without bioreactor) culture conditions. Overall, cells do not penetrate more than ˜100 μm from the surface and their growth is thus limited within this limited penetration depth for a maximal saturation value. The saturation value, while being affected by attachment modifiers (such as Hyaluronic acid, HA) or culture media volume, is nevertheless constant for each set of conditions and represents the upper boundary of static culture conditions. Graph 601 in FIG. 6A shows a custom developed mathematical modeling of empirical data sets for HA treated (represented by diamonds) and non-treated (represented by circles) pcECM matrices showing the attachment density as a function of initial seeding density. Graph 603 in FIG. 6B shows a goodness-of-fit between predicted and measured values. The cell loading capacity of HA-treated scaffolds (4.0×10⁵ cells/cm²) was significantly higher (p<0.0001) than that of the nontreated pcECM matrices (2.7×10⁵ cells/cm²). Graph 605 in FIG. 6C shows cell density changes as a function of time for low seeding densities (5×10⁴ cells/cm²). Graph 607 in FIG. 6D shows cell density changes as a function of time for high seeding densities (1.5×10⁷ cells/cm²). Graph 609 of FIG. 6E shows the effect of medium volume on cell density. Photograph 611 of FIG. 6F shows hematoxylin and eosin (H&E) staining of representative histological cross-sections of reseeded pcECM constructs that were cultivated for 21 days, under static culture conditions. Scale bar 613 in FIG. 6F represents 100 μm. For each experimental group and density there are five biological replicas (n=5). Insets in FIG. 6C-FIG. 6E show the least square means computed by two-way analyses of variance (ANOVA). * in FIGS. 6D and 6E denotes significantly different results (p<0.05).

FIGS. 7A-7L show experimental data for compartmentalized dynamic recellularization using mono-cultures of human mesenchymal stem cells (hMSCs) and human umbilical vein endothelial cells (HUVECs). These cell types are representative of a parenchymal reparative cells and pericytes (hMSCs); and blood vessel lumen coating endothelial cells (HUVECs). FIG. 7A shows a functioning perfusion chamber 701 that can be trans-located from the CO₂ incubator into a biological cabinet where sterile handling is available. Using this system, decellularized thick pcECM scaffolds 703, as shown in FIG. 7B, regain full thickness appearance after 48 hrs of perfusion to form tissue construct 705, as viewed from top in FIG. 7C and viewed from side in FIG. 7D. Photographs 707 in FIG. 7E shows H&E staining seven days post dynamic cultivation of hMSCs seeded through the bulk of the pcECM by injection. Graph 709 in FIG. 7F shows cell survival when cultivated under various physiological flow rates, using different seeding times (1.5 or 24 hrs), determined after 24 hrs of perfusion. * in FIG. 7F denotes significantly different results p<0.05. Graph 711 in FIG. 7G shows transferring of statically cultivated thick constructs (t=30 days, marked with an arrow) to further cultivation in the dynamic system exhibits a significant (p<0.05) increase in cell quantities. Dashed line represents the 95% confidence interval of the mean. Photograph 713 in FIG. 7H shows histological cross-sectional imaging of cell penetration depth. ECM fibers' autofluorescent signal 712 and cell nuclei 714 are shown (counterstained with 4′,6-diamidino-2-phenylindole, DAPI). Photograph 715 in FIG. 7I shows specific antibody staining for CD44 which suggests that the hMSCs are anchored to the pcECM through their HA receptors. Photograph 717 in FIG. 7J shows live confocal imaging (hMSCs stained with Hoechst) of the endocardial surface after 21 days of static culture which reveals densely populated surfaces in accordance with the mathematical model prediction of steady state densities. Photograph 719 in FIG. 7K shows re-endothelialization of the vascular network within the pcECM using a mono-culture of HUVEC-GFPs (human umbilical vein endothelial cells—green fluorescent protein, as indicated by 725 in FIG. 7K) forming 14 days postseeding and perfusion, which demonstrated a monolayer coating in a cobble stone-like formation 720. Photograph 721 in FIG. 7L shows cross-section staining of the GFP (green fluorescent protein) expressing cells (which may be green and as indicated by 727 in FIG. 7K) with CD31 (which may be red and as indicated by 729 in FIG. 7K) demonstrates endothelium formation within the lumen of the blood vessel. In all experiments, results represent at least 3 biological repetitions (n>3). Scale bars 723 in FIG. 7E, FIG. 7H, FIGS. 7J-7L represent 100 μm. Scale bar 725 in FIG. 7I represent 50 μm.

FIGS. 8A to 8E shows experimental data for dynamic co-culturing and re-vascularization of thick pcECM scaffolds. Online monitoring of cell culture conditions throughout the dynamic long-term co-cultivation of HUVEC-GFPs and hMSCs are shown in FIG. 8A-8B. Graph 801 in FIG. 8A shows total cell quantity and cell lactate dehydrogenase (LDH)-cytotoxicity evaluation (represented by circles and squares, respectively) as a function of time. Graph 803 in FIG. 8B shows glucose consumption and lactate production (represented by circles and squares, respectively), which represent measures for cell metabolism, as a function of time. Reduction in glucose and increase in the level of lactate are attributed to cell metabolism whereas an increase in glucose and reduction in lactate levels are the consequences of culture medium changes zeroing the measurements to their baseline level. Vertical error bars represent standard error of the mean. Horizontal error bars represent standard deviation in time. Photograph 805 in FIG. 8C shows fluorescent monitoring of HUVEC-GFPs throughout the co-culture dynamic experiment, showing live imaging of most of the large pcECM installed, including the main blood vessels at t=3. Photograph 807 in FIG. 8D shows fluorescent monitoring of HUVEC-GFPs throughout the co-culture dynamic experiment, showing live imaging of most of the large pcECM installed, including the main blood vessels at 21 days. Photograph 809 in FIG. 8E shows a zoom-in view on the white rectangle 811 appearing in FIG. 8D in which sprouting blood vessels from precoated vessels are apparent and are positive for the fluorescent signal (which may be green and indicated by 819 in FIG. 8D and 8E) due to the involvement of HUVECs in this process. The scale bars 813 in FIGS. 8C, 8D and 8E represent 2 mm. In all experiments, results represent 3 biological repetitions (n=3).

FIGS. 9A-9D show experimental data for in-vitro functional angiogenesis. Photograph 901 in FIG. 9A shows a cross-sectional overview of a small arteriole and its surrounding tissue 21 days post co-culture dynamic cultivation (2×2 mm field of view). Sprouting of new vessel-like pathways, in various stages of maturation, is subjected to interplay between the sprouting HUVEC-GFPs and hMSCs (CellVue® Claret) at the periphery of the supply arterioles. HUVEC-GFPs may be green and hMSCs (CellVue® Claret) may be red as indicated by 921 and 919 respectively in FIG. 9B. Photograph 913 in FIG. 9B represent higher magnification of the rectangle 903 marked in FIG. 9A. Photograph 915 in FIG. 9C represent higher magnification of the rectangle 905 marked in FIG. 9A. FIG. 9B and FIG. 9C show different stages of cell sprouting. At the initial stages of sprouting, hMSCs seem to concentrate around the base of the newly formed vessel in FIG. 9C, followed by eruption of an endothelial cell front accompanied by fewer hMSCs as also demonstrated in FIG. 9B. Photograph 907 in FIG. 9D also shows eruption of an endothelial cell front accompanied by fewer hMSCs. Scale bars 917 in FIGS. 9A, 9B and 9C represent 200 μm. In all experiments, results represent 3 biological repetitions (n=3).

In the following, experimental data will be described.

Recently, cardiac acellular extracellular matrix (ECM) from rats and pigs were successfully isolated. The ECM was proposed as an ideal scaffolding biomaterial for cardiac regeneration. The decellularization of full-thickness porcine cardiac ECM (pcECM) may be potentially advantageous, over other tissues and species, as it highly resembles the human ventricular wall in structure, size, and composition while its production and availability can be easily controlled through stringent quality control over its source. This study aimed to strengthen the ability to support such a platform, demonstrate the potential of this thick pcECM scaffold, and evaluate its long-term cell support and the ex vivo promotion of new blood vessel generation. For these purposes, a unique bioreactor system was designed and custom built, which may enable the long-term compartmentalized cocultivation of various stem and progenitor cells within the thick pcECM construct under dynamic physiological-like conditions. Cocultures of human umbilical vein endothelial cells (HUVECs) and human mesenchymal stem cells (hMSCs) were used herein as a proof-of-concept to demonstrate the inherent vasculature functionality and its ability to support the ex vivo repopulation of the thick tissue construct's bulk. Furthermore, a simple methodology was developed to statically determine the pcECM cell holding capacity, predicting a maximal cell density resembling that of native myocardium. Taken together, the study may demonstrate for the first time the possibility of reconstructing a functional vascular tree ex vivo, which supports compartmentalized recellularization of thick myocardial-like tissue constructs. The study may suggest an alternative and important approach to cardiac tissue engineering, which is based on preserving a connectable inherent vascular tree within the biomaterial scaffold that might facilitate future survival and function of reseeded constructs upon transplantation.

In the following, materials and methods according to various embodiments will be described.

In the following, preparation of pcECM matrices for static and dynamic culturing according to various embodiments will be described.

Porcine left ventricular full-thickness slabs (˜10-15 mm) were perfused and decellularized. For static cultivation, thick pcECM matrices were placed on standard culture plates and cut with a sterile 8 mm punch (unless stated differently). Matrices were transferred into 96-well plates, epicardial surface facing downwards. For dynamic cultivation, pcECM matrices were cut using a scalpel into ˜25×75×15 mm slabs containing the perfusion entry catheter already sutured in place (24-gauge, 8 cm long; Biometrix™). Ethanol disinfected catheters (20 min in 70% ethanol) were sutured using a sterile suturing thread (5/0 nonabsorbable thread) to the other side of the construct for drainage. Large leaks, if detected, were shunted by additional suturing. Before cell seeding, matrices of either cultivation method were washed with ethanol 70% (1×30 min, 1×2 and 1×12 h) followed by at least three washes with phosphate-buffered saline (PBS; 3×30 min), immersion in complete culture media for 12 h, and air-drying in a sterile hood for 2 h.

In the following, cell isolation and cultivation according to various embodiments will be described.

Bone marrow hMSCs were purchased from Lonza and cultured in humidified incubator at 37° C. and 5% CO2 using alpha modified Eagle's medium (α-MEM; Biological Industries) supplemented with 20% fetal bovine serum, 1% Pen-Strep, and 0.4% Fungizone®. HUVECs stably expressing GFP (HUVEC-GFP) were kindly donated by Prof. Gera Neufeld (Technion, Faculty of Medicine) and cultured on gelatin-coated plates (0.2% gelatin in PBS, 37° C., >4 h; Sigma-Aldrich™) with M199 culture media supplemented with 20% fetal calf serum, 1% Pen-Strep®, and 0.4% Fungizone (Life Technologies). Basic fibroblast growth factor (10 ng/mL) was added to plates of both cell types every other day. Whenever HUVECs and hMSCs were cocultured, α-MEM was used. Human embryonic stem cell-derived cardiomyocytes (hESC-CM) were expanded, differentiated, and statically cultivated on the pcECM according to the following protocol.

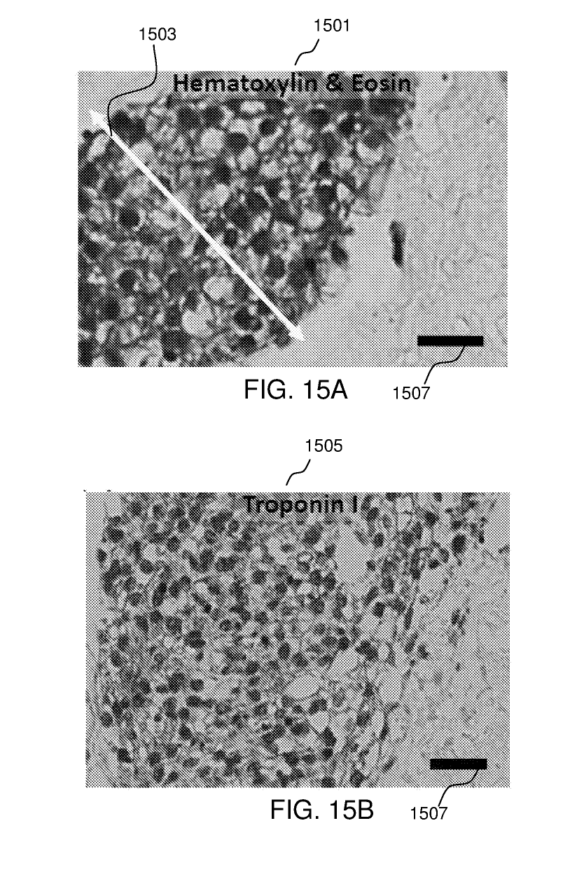

hESC-CM were expanded and differentiated using an intermittent rocker system. Following differentiation 1.1×10⁷ hES3-CM cells in RPMI (Roswell Park Memorial Institute) medium containing 2% Pen-Strep®, 0.8% Fungizone®, B27-insulin (2%, Life Technologies) and the ROCK (Rho kinase) inhibitor Y27632 (Calbiochem, Merck-Millipore, Singapore) were seeded on each thick pcECM scaffold (n=3, diameter—1.5 cm) for 90 min followed by cultivation in 10 ml culture media changed every other day for up to 23 days. Beating was documented using the EVOs phase contrast microscope (Advanced Microscopy Group, Life-Technologies, Carlsbad, Calif.) equipped with a 4× lens (Olympus) and recorded using the manufacture's provided software (Advanced Microscopy Group). At termination, seeded matrices were fixated in 4% paraformaldehyde (PFA) over-night followed by paraffin embedding, sectioning (5 μm), histochemical (H&E) and immuohistochemical (IHC) staining (Advanced Molecular Pathology Laboratory, A*Star) for Troponin I. For IHC stains, an antigen retrieval step at pH=9 (40 min, 100° C., Bond™ epitope retrieval solution 2, Leica-microsystems, Germany) was performed followed by endogenous peroxidase blocking (45 min, 3.5% H₂O₂), 10% goat serum blocking for lhr and primary antibody incubation with a mouse monoclonal anti human Troponin I antibody (ab19165, abcam, 1:200). Anti-mouse poly HRP-IgG in 10% animal serum was then added for 5 min, color was developed with Bond™ Mixed DAB refine solution (Leica-microsystems, auto-stainer reagents, Germany) and counterstained with hematoxylin for 5 min prior to dehydration and mounting in synthetic mounting media. Photograph 1501 in FIG. 15A shows Hematoxylin & Eosin histological stain (H&E) of beating pcECM repopulated with hESC-CM and statically cultivated for 23 days. Nuclei are counterstained with Gill's hematoxyilin. Scale bars 1507 in FIGS. 15A and 15B represent 20 μm. The arrow 1503 points for the span of repopulated pcECM penetration depth (100 μm). Photograph 1505 shows Trooponin I immunohistochemical stains as a marker of the contraction machinery.

In the following, assessment of pcECM cell support according to various embodiments will be described.

To evaluate the pcECM maximal cell capacity under static culture conditions, mathematical modeling was employed. This model was based on empirical data and verified by an additional set of experiments in which the quantity of cell adhesion foci was artificially changed, for example, through cross-linking of HA to the pcECM matrices, to investigate the model sensitivity.

The mathematical model may be developed according to the following.

Eq. 1 can be used to describe cell adhesion to the pcECM. [S] denotes the scaffold surface density of unbound (free) cell adhesion foci (CAF) as CAF/cm²; [C] represents the surface density of unbound cells; and [SC] represents the density of cell-bound CAFs. K_{eq} represents the equilibrium constant of the cell binding to the CAF within the seeding time period permitted for cell attachment.

$$[ S ] + [ C ] {}^{{K}_{eq}} [ SC ] . \qquad Eq . 1$$

Assuming first order kinetics, Eq. 2 can be stipulated to describe the K_{eq} constant:

$${K}_{eq} = \frac{[ SC ]}{[ S ] \cdot [ C ]} . \qquad Eq . 2$$

Denoting the density of bound CAFs (SC) as a variable ‘x’, given known initial seeding cell densities (C₀), and a finite, yet unknown, CAF density (S₀), Eq. 2 could be re-written as follows:

$${K}_{eq} = \frac{[ SC ]}{[ S ] \cdot [ C ]} = \frac{x}{( {S}_{0} - x ) \cdot ( {C}_{0} - x )} . \qquad Eq . 3$$

After algebraic rearrangement, a quadratic expression of Eq. 3 can be deduced as follows:

K_{eq}x²−x(K_{eq}S₀+K_{eq}C₀+1)+KS₀C₀=0 Eq. 4

The roots of this quadratic equation (Eq. 4) can be found according to Eq. 5 below:

$${x}_{1 , 2} = \frac{\begin{matrix}( {K}_{eq} {S}_{0} + {K}_{eq} {C}_{0} + 1 ) \pm \\ \sqrt{{( {K}_{eq} {S}_{0} + {K}_{eq} {C}_{0} + 1 )}^{2} - 4 {K}_{eq}^{2} {S}_{0} {C}_{0}}\end{matrix}}{2 {K}_{eq}} = [ SC ] . \qquad Eq . 5$$

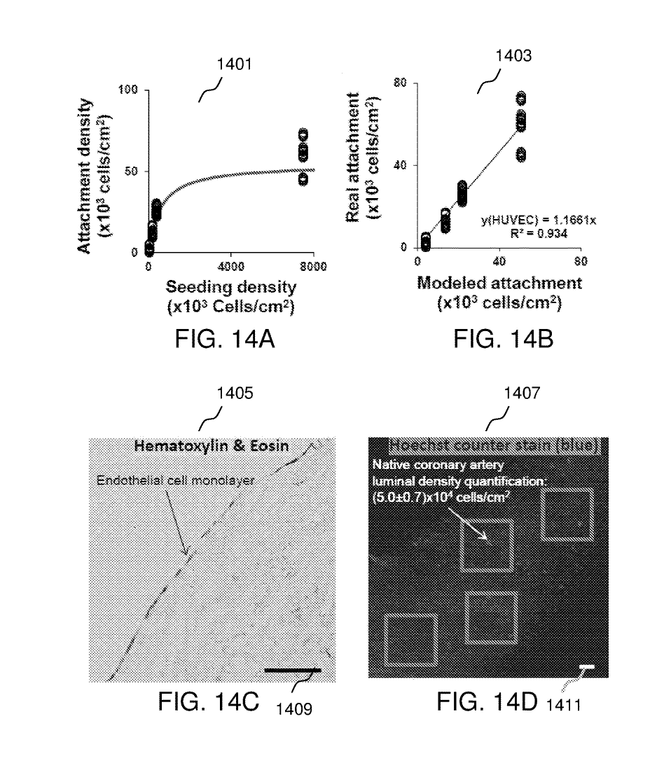

According to Eq. 5, the density of matrix bound cells at steady state [SC=x] can be calculated and expressed as a function of K, S₀ and C₀. With increasing quantities of C₀ towards a saturated SC value (i.e. C₀>>S₀→SCₛₐₜᵤᵣₐₜᵢₒₙ≈S₀) S₀ could be estimated by x representing the maximal matrix cell-holding capacity per cm². Given a known set of C₀ (different seeding densities) and SC values (measured through AlamarBlue™), S₀ can be optimized (Microsoft Excel™ solver) to achieve the best fit (least squares method) of empirical data to model predictions (Eq. 5). Approximate S₀ values were used as boundary conditions (estimated by plotting attached cell density as a function of the seeded cell density). Thus to delineate S₀, hMSCs (FIGS. 6A-6E) or HUVECs (FIGS. 14A-14B) were seeded on the pcECM in different densities (5×10⁴, 2×10⁵, 4×10⁵ and 1.5×10⁷ cells/cm² for hMSC and 5×10⁴, 2×10⁵, 4×10⁵ and 7.5×10⁶ cells/cm² for HUVECs) and allowed to adhere for 90 min before 2 ml of complete culture media were added and incubated for an additional 24 hrs to achieve steady state. To evaluate cell attachment density, the matrices were transferred to fresh 24-well plates containing 2 ml of culture media supplemented with 10% (v/v) AlamrBlue™. Cell quantities were determined against the appropriate calibration curve using five biological replicates.

The measured attachment densities (SC) were plotted as a function of the modeled density (Eq. 5, FIGS. 6A-6E, FIGS. 14A-14B).

The mathematical model may be verified according to the following.

Two methodologies were used to evaluate the validity of this model. The first methodology involved the artificial modification of the pcECM adhesion foci quantity, testing model sensitivity to artificially modified S₀ values. In a second methodology, the monitoring of cell proliferation for long-term static cultivation was performed, allowing sufficient time for cell proliferation and reaching the theoretical maximal matrix capacity, proving its long-term cell support ability (>3 days).

In the first methodology, HA+EDC-NHS (hyaluronic acid+ethyl(dimethylaminopropyl)-carbodiimide-N-Hydroxysuccinimide) treated matrices were re-seeded with the same experimental densities and modeled again according to the steps above. These matrices served as a distinct empirical set aiming to prove model sensitivity to changes in adhesion foci quantities and suggest applicability of this model for other scaffolding materials as well.

Acellular pcECM constructs of both HA+EDC-NHS and non-treated matrices seeded with two representative hMSC densities (5×10⁴ and 1.5×10⁷ cells/cm²) were monitored for 21 days (n=5 samples per group) with AlamrBlue™ (at t=1, 7, 14 and 21 days post seeding).

In the second methodology, to evaluate the effect of culture medium volume on the proliferation of reseeded cells, pcECM matrices were reseeded with equal densities of 2×10⁵ hMSC/cm² (just below the maximal cell capacity predicted by the model for both treated and non-treated matrices) and divided into two culture groups (n=5 samples per group) having different culture medium volumes (2 or 10 ml/well), replaced every other day. Cell density was determined using AlamrBlue™ at t=1, 7, 14 and 21 days post seeding. At t=21 days, the experiment was terminated, matrices were documented and histological assessment was performed using standard H&E (Sigma, USA) staining.

Static seeding and cultivation of hMSCs on the pcECM scaffolds for the mathematical modeling were performed. Briefly, pcECM scaffolds (8 mm in diameter) were immersed in 96-well plates containing α-MEM complete culture media for 12 h in cell-culture conditions. Before seeding, media was removed and scaffolds were left to partially dry for 2 h. HMSCs were resuspended in complete α-MEM, to a final concentration of 1.4×10⁴ cells/μL, seeded on the matrices with increasing cell densities (5×10⁴, 2×10⁵, 4×10⁵, and 1.5×10⁷ cells/cm² in quintuplicate per each density), and cultivated for 21 days. Seeding was performed through pipettation by slowly administering the appropriate cell suspension volume (as per the cell quantities detailed above) onto the center of the scaffolds. Seeded scaffolds were preincubated in culturing conditions for 90 min, previously reported as the optimal seeding time, and transferred to new plates for cultivation. Unless mentioned otherwise, each reseeded matrix was incubated in 2 mL of hMSC complete growth media, replenished every other day. Similar experiments were also performed with HUVECs.

In the following, a bioreactor system design and setup according to various embodiments will be described.

A schematic description of the bioreactor design and setup used throughout these experiments is presented in FIG. 3. The “heart” of the system is the perfusion chamber (in other words the bioreactor module) 200. This custom-built chamber 200 holds the matrices in place (marked by a mesh in FIG. 2B) under sterile culture conditions, and it enables both pulsatile flow perfusion and mechanical and electrical stimulation, mimicking the heart physiological environment. A glass cover (in other words the bioreactor housing, 340) enables online monitoring and imaging under sterile culture conditions. The chamber 200 is located within a standard CO₂ incubator 342 (marked by gray-shaded square in FIG. 3), maintaining a temperature of 37° C. throughout the system. A MasterFlex™ peristaltic pump 324 is used to pump the culture media from a glass medium reservoir 322 (Radnoti LLC) to the perfusion chamber 200. A silicon tube oxygenator 328 (Radnoti LLC) and a no-return check valve 326 (Cole Parmer), located between the pump 324 and the perfusion chamber 200 ensure maintenance of proper oxygen levels. A second channel 334 for drainage of excess culture media from the bioreactor module 200 (marked by a dashed line) pumps the media back into the reservoir 322. A third low volume channel 332 (dotted line) is used to bypass the reservoir 322 when concentration-dependent measurements are taken to assess cell quantity and metabolic state throughout long term experimentation.

According to various embodiments, perfusion chamber materials may be chosen to enable maximal biocompatibility. The baths, matrix holders and base plate may be made of polyether-etherketone (PEEK), the cover may be made of glass and all connectors may be made of food grade stainless steel. All tubing used may be made of medical grade silicone 1.5×3 mm tubing and all tubing connectors may be luer connectors (Cole Parmer, Vernon Hills, Ill.). Three tube lines may be used, one for feeding, a second for drainage and a third for low volume applications (FIG. 3). The tubes entering and exiting the pump head may be different than the rest of the tubing as these determined the flow ratio between all the channels, given a particular pump rotating speed. The only non-silicone tubing may be that leading from the oxygenator to the perfusion bath (Tygon® R-3603, 0.8×2.4 mm) as silicone is gas permeable.

For system installation, a standard CO₂ incubator may be located next to a biological safety cabinet (BSC) to enable smooth passaging of the perfusion chamber from the incubator to the hood and back, allowing aseptic handling. Prior to installation the oxygenator, perfusion chamber (with its cover open) and reservoir may be autoclaved, sprayed with ethanol 70% and inserted into the biological hood for 15 min. The perfusion baths may be connected with ethanol-disinfected 24 G catheters to the entry port and the glass cover closed. An air filter may be connected to the air entry port from the outside (0.22 μm, Millipore, Billerica, Mass.) using a standard sterile infusion lengthening tube (50 cm manometer line M/F, Biometrix, Jerusalem, Israel). Tygon® tubing may be connected to the oxygenator and the perfusion chamber. The oxygenator may be connected to the ethanol-disinfected check valve and to the entry and exit air filters (Millipore, UK). The end connectors of the pre-installed tubing lines may be closed at each end with luer combi M/F stoppers (Biometrix), sprayed with ethanol, inserted into the biological hood, and connected to their relevant matching connectors in the oxygenator and the perfusion chamber. The air tubing may be connected to the oxygenator and to a standard “fish tank” air pump actively pushing the incubator air through the oxygenator. Subsequently, the perfusion pump may be activated.

For system disinfection, the system may be perfused with 500 ml of 70% ethanol for 30 min. The returning line may not be allowed to enter into the reservoir; instead, it may be directed to a waste container and discarded. This may be followed by circulation of an additional 500 ml of ethanol for 2 hrs, thereafter replaced with fresh 70% ethanol and circulated overnight. The system may then be aseptically installed with dynamically prepared pre-cut pcECM matrices, followed by perfusion of the culture media overnight and air drying in the hood for 90 min prior to cell seeding.

In the following, cell seeding and dynamic cultivation standard operating procedure according to various embodiments will be described.

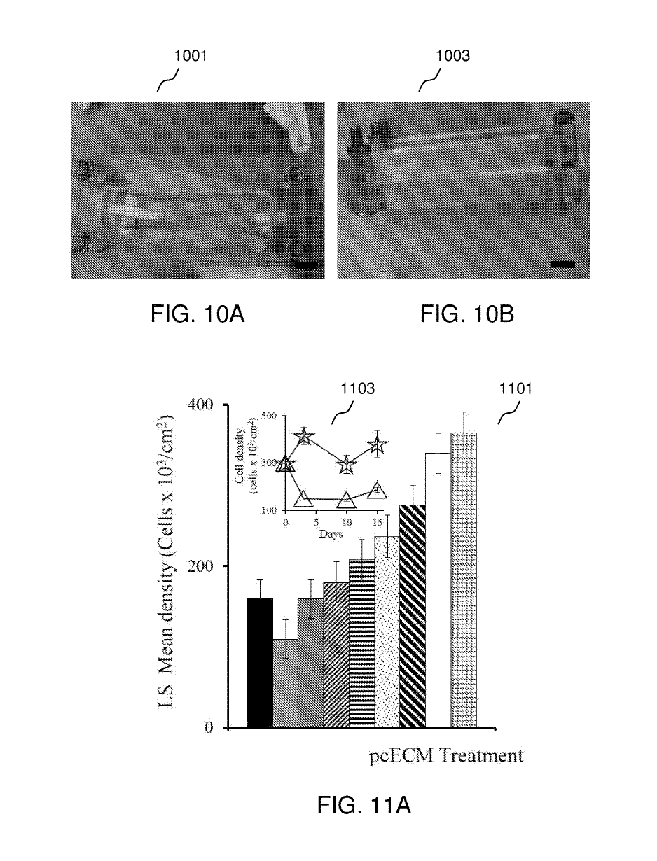

For HUVEC-GFP cell seeding, sterile matrices were removed from the perfusion chamber (in the BSC) and transferred into custom-built and ethanol-disinfected (70% ethanol, 30 min) seeding frames (FIGS. 10A and 10B). For seeding, 1 ml solution of 1×10⁷ cells/ml was injected through the entry catheter with a 2 ml syringe and incubated for 60 min in the hood covered by a sterile 20 cm plate cover. During this 60 min incubation, the frames were rotated several times. Seeded matrices were transferred back into the perfusion bath (epicardial side facing upwards), inserted into place and the baths filled with 60 ml of complete HUVEC culture media per bath.

MSC cells were seeded by either injection through the bulk cavities or pipettation on the endocardial surface of dynamically prepared matrices. Injection was used to deliver cells deeper into the matrix for initial assessment experiments or when co-culture experiments were performed with HUVEC-GFPs. Injection was performed throughout the matrix bulk using a 25 G syringe in multiple locations until a uniformly inflated matrix appeared (1×10⁶ cells/ml in 10 ml culture media: total 1×10⁷ cells). Pipettation on the endocardial surface (nitrocellulose treated) was used to enable static culture conditions reaching cell density steady state for 30 days. The static cultures were then transferred into and cultivated in the bioreactor system for an additional period of 14 days to evaluate the effect of dynamic culturing—assessing cell penetration (using histology and specific antibody staining for CD44 (mouse anti human, Cat. No. 555476, BD Biosciences, San Jose, Calif.), and proliferation (using Alamar Blue™).

Unless otherwise stated, dynamic cultivation was based on the low volume cycle (120 ml per construct). Complete media was replenished every other day. Samples of 100 μl each were taken prior to medium exchange of both old and new culture media to check for contamination. Of these samples, 50 μl were suspended in duplicate in 96-well plates containing 50 μl of liquid highly nutritious general-purpose growth medium for fastidious microorganisms (brain heart infusion broth, BHI, Sigma, USA). Samples were incubated overnight at 37° C. under shaking (250 RPM) and were read the next day at 600 nm compared to blank containing sterilized media and BHI mixtures.

In the following, determination of the dynamic cultivation parameters according to various embodiments will be described.

To determine the optimal seeding time and perfusion flow rate, acellular pcECM constructs were seeded with hMSCs, installed in the perfusion chamber baths (in other words the bioreactor module), covered with 60 mL of complete MSC culture media, and incubated for 1.5 or 24 h before starting perfusion, allowing cell attachment. To measure cell viability, 5% AlamarBlue™ (Invitrogen™) in complete culture media was perfused for 24 h (low volume cycle, bypassing the reservoir) at 40 or 80 mL/min. Media samples (300 μL) were taken throughout the culture via the sampling port, the AlamarBlue fluorescence intensities were measured (Ex: 530/25 nm, Em: 595/35 nm), and cell numbers were calculated thereof versus the appropriate calibration curve. The survival rate determined 24 h postcommencement of perfusion was estimated by dividing the AlamarBlue determined cell quantities with the initial seeding quantity (1.4×10⁷ cells/construct). In another experiment, the optimized cultivation parameters (1.5 h seeding time, 120 mL/construct perfused at up to 40 mL/min and replenished every other day) were assessed in terms of hMSC support for up to 7 days. Constructs were cross sectioned and stained with hematoxylin and eosin (H&E). Representative images are presented out of a total of three constructs processed and at least three histological cross sections per construct.

In the following, compartmentalized recellularization according to various embodiments will be described.

The optimized dynamic cultivation parameters were further assessed in terms of their effect on constructs, which were precultivated in steady state densities under static culture conditions. Thus, hMSCs (3×10⁵ cells/cm²) were seeded onto the endocardial surface of 25×70×15 mm acellular pcECM slabs, statically monocultured for 30 days, and transferred to the bioreactor system 300 for dynamic culturing for an additional period of 14 days. Cell viability (AlamarBlue), density and penetration toward the feeding blood vessels (histology) were assessed. Alternatively, HUVECs stably expressing GFP were resuspended (10×10⁶ cells/mL) in 0.2% gelatin in complete M199 culture media and seeded through the vascular network. Fourteen days postseeding live imaging was performed through confocal microscopy to evaluate the extent of vascular network coating, followed by histological cross section and staining with CD31.

Histological analyses were performed on cryo sections (10 μm thick, using a CM3050 cryostat, Leica, Wetzlar, Germany) to visualize cell morphology and penetration depth of both static and dynamically cultivated constructs. Unless otherwise stated, representative images are presented out of a total of at least three blocks per construct and are based on several histological cross-sections (n>3) taken from different locations on the Optimal Cutting Temperature (OCT) blocks. With the exception of HUVEC-GFP seeded matrices (PFA 2%—5 hrs, PFA 1%—2 hrs and 30% sucrose 48 hrs), samples were not fixated prior to freezing. Cross-sections were methanol (−20° C.) fixated to the slides and either H&E stained (Sigma, St. Louis, Mo.) or mounted with DAPI (Fluoromount G, Southern Biotech, Birmingham, Ala.) for fluorescent imaging of HUVEC-GFPs and/or MSCs stained with ClaretVue™ dye (Sigma, St. Louis, Mo.). For protocols involving static mono-cultures of hMSC, non pre-stained hMSCs were used, which were counter stained with DAPI following histological sectioning and imaged adjacent to the ECM fibers fluorescent signal (FIG. 7H to 7L and FIG. 13).

For specific cell staining, blocking was performed with 5% fetal bovine serum (FBS) in phosphate-buffered saline (PBS) for 1 hr at room temperature, followed by incubation in 4° C. overnight with mouse anti-human CD44 (555476, BD Biosciences, San Jose, Calif.) or rabbit anti-rat CD31 (sc-3806, Santa Cruz Biotechnology, Dallas, Tex.) primary antibodies diluted (CD44: 1:25; CD31: 1:50) in PBS containing 3% FBS overnight. Sections were washed 3×5 min in PBS, followed by incubation with a secondary antibody (goat anti-mouse FITC, 1:300, Sigma F8521, St. Louis, Mo. or donkey anti-rabbit PE, 1:100, sc-3745, Santa Cruz Biotechnology, Dallas, Tex.) for 1 hr (room temperature). Slides were then washed 3×5 min with PBS, mounted using DAPI containing fluoromount G (water based) and covered with cover slides that were glued using a transparent nail polish. All fluorescent imaging was performed using an inverted confocal microscope (LSM700, Carl Zeiss Germany) with an EC Plan-Neofluar 10x/0.30 M27 air lens or using a fluorescent inverted microscope (Nikon, Ti-S model, Japan) equipped with a 20× air lens.

In the following, dynamic compartmentalized cocultivation of surface/bulk recellularized (hMSCs) and blood vessel lumen re-endothelialized (HUVEC) acellular pcECM will be described.

According to various embodiments, the first cell type may be seeded in an interior of a vessel of an inherent vascular network of a scaffold. The scaffold may be rotated to coat the vessel with the first cell type. The scaffold may then be mounted onto the bioreactor module 200. The vessel of the inherent vascular network of the scaffold may be connected to the inlet 206 of the bioreactor module 200. The scaffold may be incubated in the bioreactor module 200 for a period of time before perfusion via the inlet 206 through vascular network of the scaffold commence. A second cell type may be seeded on an exterior surface of the scaffold. Seeding of the second cell type may be via injection into the exterior surface of the scaffold or by pipettation on the exterior surface of the scaffold. In this method, perfusing of the scaffold via the inlet 206 of the bioreactor module 200 with culture medium may facilitate compartmentalized co-cultivation of the first cell type and the second cell type in different niches of the cultivated tissue.

According to various embodiments, a in-vitro method for tissue cultivation may include seeding an interior of a vessel of an inherent vascular network of a scaffold with a first cell type, seeding an exterior surface of the scaffold with a second cell type, and perfusing through the inherent vascular network of the scaffold with culture medium may facilitate compartmentalized co-cultivation of the first cell type and the second cell type in different niches of the tissue. The method may further include connecting the vessel of the inherent vascular network of the scaffold to an inlet of a bioreactor module 200 as described herein, wherein perfusing through the inherent vascular network of the scaffold comprises perfusing via the inlet 206 of the bioreactor module 200.

According to various embodiments, the scaffold may be perfused initially with a first culture medium. The first culture medium may be gradually replaced with a second culture medium to ensure cell acclimation to the culture media change towards co-culture conditions. The first culture medium may include M199 or EGM2 (Endothelial Growth Medium-2) medium. The second culture medium may include culture medium for supporting the co-culture conditions, such as α-MEM, RPMI or mTEASER or any other equivalent medium.

According to various embodiments, growth factors and cytokines such as human recombinant vascular endothelial growth factor (VEGF) basic fibroblast growth factor (bFGF) or any other factor (cell type dependent) to the culture medium which diffusion can cause cell survival, proliferation, polarization, migration and integration may be added to the culture medium. The scaffold may be a decellularized extracellular matrix with an inherent vascular network preserved.

Long-term coculture of HUVEC-GFPs (in other words a first cell type) and MSCs (in other words a second cell type) was studied for 21 days. HUVEC-GFPs were seeded into the inherent vasculature of acellular pcECM slabs as previously published with slight modifications. Photographs 1001 and 1003 of FIG. 10A and FIG. 10B show examples of seeding frames setup. Photographs 1001 and 1003 shows clamped pcECM matrix from its epicardial surface and from the side respectively as used for HUVEC seeding inside the main vasculature. Re-endothelialized matrices (n=3) were mounted onto the perfusion chamber and incubated for 1.5 h before starting perfusion (up to 40 mL/min) with complete M199 media, which was replenished every other day. For coculturing with hMSC, the culture media was gradually replaced, during the first week, with complete α-MEM. One week after re-endothelialization (t=8 days), prestained hMSCs (Claret-CellVue™; Sigma-Aldrich) were seeded onto the same matrix by injection. Seven days after MSCs were included in the coculture (t=15 days), human recombinant vascular endothelial growth factor (VEGF; Sigma-Aldrich) was added (3 ng/mL) and replenished every other day for an additional week.

Online monitoring was conducted throughout these experiments, to assess cell viability, metabolism, process cytotoxicity, and maintenance of physiological pH. Cell viability was determined using dynamic AlamarBlue measurements on days 1, 6, 10, 15 and 21. Measurements of glucose (Freestyle™; Abbott Laboratories) and lactate (Lactate scout; EKF Diagnostics) were performed throughout the study. Cytotoxicity was evaluated in culture media 24 and 48 h after media replacement by measuring the activity of lactate dehydrogenase (LDH) using an LDH cytotoxicity detection kit (Roche), according to the manufacturer's instructions. LDH measurements represent the excess measured quantity after blank substitution (supplemented fresh culture media not exposed to cells kept for the same time duration within the same culture conditions). Background absorbance was eliminated by subtracting reads at 620 nm from the actual reads at 492 nm. Media pH was measured throughout the process using a standard narrow-electrode pH meter (Seven Easy™; Mettler Toledo) and maintained at physiological levels (7.2-7.4, data not shown) by changing the incubator's CO₂ concentration.

HUVEC-GFPs were live-imaged within the vascular network through the perfusion chamber glass cover on days 3, 10, and 21 using Olympus SZX16 (Olympus Corporation) binocular fluorescent microscope equipped with 0.8× dry macro-lens with numerical aperture of 0.3 and a working distance of 81 mm. Exposure times were coordinated with those previously determined for blank matrices (before seeding, data not shown). On day 21 the matrices were removed from the bioreactor and subjected to fluorescent histological cross-section analyses and imaged with LSM700 (Carl Zeiss).

In the following, statistical analysis according to various embodiments will be described.

A pilot screening experiment (n=6 biological replica per treatment group) was used to verify results' normal distribution and estimate sample size required based on 70% of measured effect size given conventional α=0.05 and minimal power of 80%. Outliers were excluded based on the Mahalanobis D² method. Border zone cases were evaluated by the “Jacknife” criteria as well. For all experiments matrices were randomly allocated for each group. Unless otherwise specified, results are expressed as the mean±standard deviation of either five or three biological repetitions per each experimental group in static (n=5) and dynamic (n=3) studies. Statistical significance in the differences of the means at individual time point experiments was evaluated by one-way analyses of variance (ANOVA) and Tukey's HSD test for multiple comparisons. Two-way ANOVA with Tukey's HSD post hoc correction for interaction and α-level adjustment for multiple comparisons was used to test the statistical significance of differences among groups through time. Particular contrast tests on individual treatment effect versus control based on least square mean estimates per group were performed to calculate the p-value. In all comparisons, p<0.05 was considered significant. Statistical analyses were done using JMP 6.0 statistical software (SAS™).

In the following, assessment of pcECM cell support and capacity according to various embodiments will be described.

Several treatments were assessed for their ability to enhance cell adhesion on the surface of the pcECM, presumably by modifying the cell adhesion foci. These treatments included chemical cross-linking of ECM fibers using an ethyl(dimethylaminopropyl)-carbodiimide:N-Hydroxysuccinimide (EDC:NHS) sequence; collagen binding with a 14-amino-acid-long collagen binding peptide (CBP) containing the Arg-Gly-Asp (RGD) sequence (RGD-CBP peptide sequence: RGD-CBPSCQDSETRTFY, Sigma); and matrix enrichment with representative sulfated (heparan sulphate, HS) and non-sulfated (hyaluronic acid, HA) glycosaminoglycans (GAGs). In addition, EDC-NHS, known to create amide bonds between adjacent amino acids, was tested either alone or in combination with HA, HS or RGD-CBP. The triple combination of EDC-NHS, RGD-CBP and HS was also tested to assess additive or synergistic effects. Nitrocellulose, previously reported to facilitate cell adhesion to biomaterials via protein absorption, was also evaluated. Non-treated pcECM matrices served as controls. Each group was tested in five biological replicates.

Decellularized pcECM scaffolds were prepared. For pcECM treatment, each well in a 96-well plate was filled with 200 μl of solution comprising: an EDC-NHS solution prepared from 0.5 mg/ml 1-ethyl-3-dimethylaminopropyl carbodiimide hydrochloride (EDC, Sigma) and 0.7 mg/ml N-hydroxysuccinimide (NHS, Sigma) dissolved in phosphate buggered saline (PBS) solution. The pcECM specimens were allowed to react with the EDC/NHS solution for 15 min, followed by several washes with double distilled water (DDW) to remove excess EDC/NHS. An RGD-CBP solution was prepared by dissolving either rhodamine labeled (when applicable) RGD-CBPSCQDSETRTFY or a non-labeled peptide equivalent (prior to cell cultivation, Sigma, 1 mg/ml) in PBS. The specimens were blocked with 5% FBS at room temperature for 30 min followed by soaking in the RGD-CBP solution for 2 hrs and DDW rinses. HS and HA solutions (Sigma) were prepared using GAG to matrix ratios of 0.1 mg HS and 1 mg HA per mg dry matrix weight, respectively. Matrices were incubated for 2 hrs and then washed several times with distilled water. Decellularized pcECM scaffolds were also treated with nitrocellulose. PcECM matrices were immersed in a 5 ml volume of 0.1 cm²/ml of nitrocellulose sheet (Bio-Rad, Hercules, Calif.) in MeOH for 24 hrs and subsequently washed extensively in sterile PBS containing 2× antibiotic concentration (2% Pen-Strep® and 0.8% Fungizone®) for 1 hr. All specimens were then immersed in complete hMSC culture media for two hours, air dried for 90 min and seeded with 3×10⁵ hMSC/cm² in 45 μl of culture media per specimen. Cells were allowed to adhere to the scaffolds for 90 min prior to the addition of culture media to the plates. The viability of the seeded hMSCs cells was evaluated using AlamarBlue™ according to the manufacturer's protocol after 3, 7 and 14 days. The modified samples that revealed the highest viability were further subjected to histological analysis using hematoxylin and eosing stain (H&E).

Four treatments corresponding to several different factors affecting cell matrix binding were screened for their potential enhancement of the quantity of matrix cell-anchorage sites. The amount of fiber cross-linking is directly related to scaffold compliance and hence to providing physical cues that might be required for cell adhesion and proliferation. The RGD sequence has been widely used in tissue engineering and drug delivery, as it is the recognition site of cell integrin mediated collagen binding. GAGs have been reported to provide an equally important and alternative binding system to integrin mediated RGD binding. Finally, nitrocellulose is frequently used in several commercial applications and was previously reported to facilitate cell adhesion through protein adsorption. These four treatments were conducted either solely or with various combinations thereof.

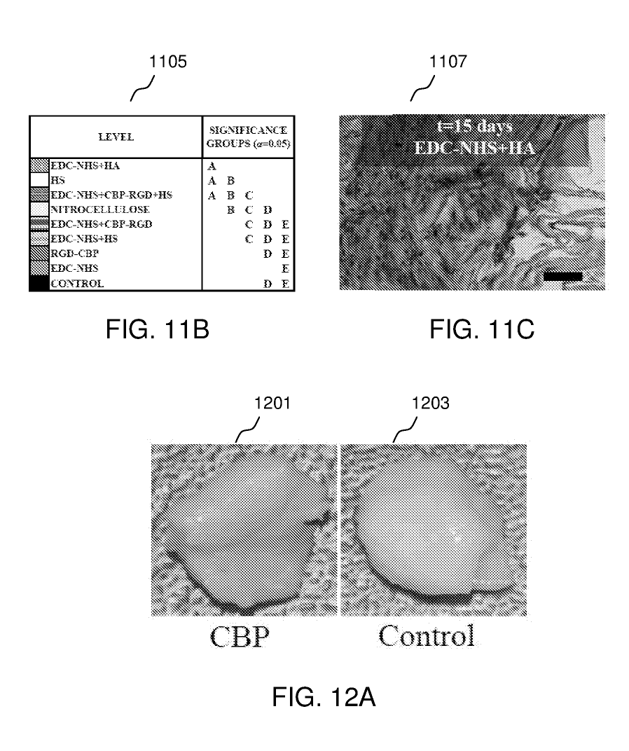

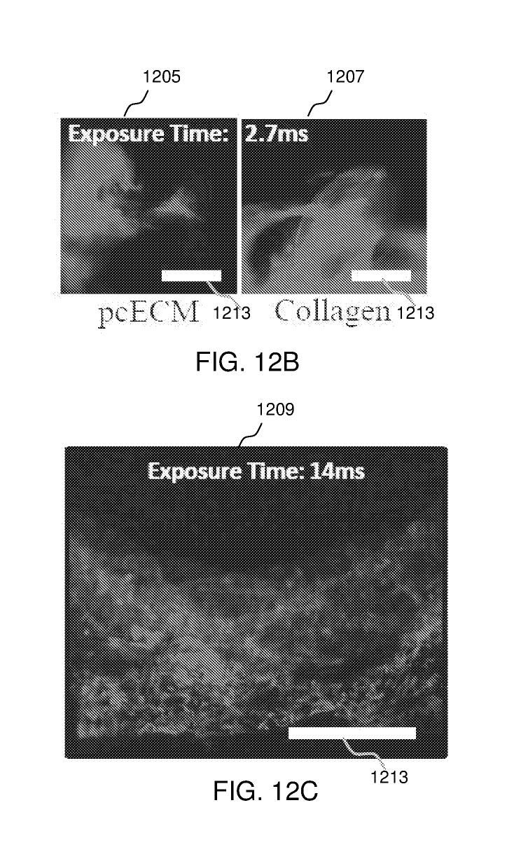

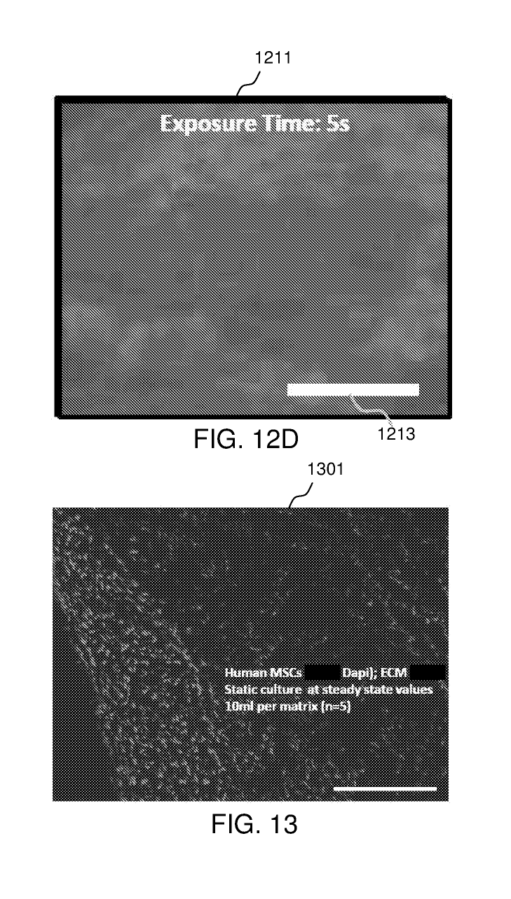

Both the nitrocellulose treatment as well as the conjugation of sulfated and non-sulfated GAG demonstrated significant cell support (p<0.05, ANOVA, FIGS. 11A-11C), suggesting that the major component required for cell adhesion to the acellular thick pcECM matrix is GAG and its respective receptors, and not cross-linking or lack of structural integrity and RGD sequences. Furthermore, collagen binding site integrity was demonstrated by incubating acellular thick pcECM matrices with fluorescently labeled RGD containing collagen-binding protein (FIGS. 12A-12D), as compared to a positive control of commercial collagen Type 1 (Sigma, St. Louis, Mo.). The results indicated that RGD-CBP binds successfully to both commercial collagen and thick pcECM, even after thorough washes with 5% BSA containing wash buffer (10×5 min each), suggesting specific peptide—ECM interaction and the preservation of the collagenous network structural integrity.

When hMSC seeding was performed at higher than maximal cell holding densities (i.e. >2.7×10⁵ cells/cm²) steady-state static culture penetration depth to the pcECM was reached starting from minimum three days post seeding. This penetration depth appeared to be limited to the first 100 μm from the surface (FIG. 13); with similar penetration depth observed also when hESC-CM (FIGS. 15A-15B) and human ventricular fibroblasts (data not shown) were cultivated on the pcECM. However, this parameter value was also dependent on the cell type used, as employing the same histological sectioning methodology, revealed that endothelial cells preferred to form a monolayer coating of the surface and did not penetrate into the pcECM any further. Hence when assessed through our developed mathematical modeling (R²=0.93) endothelial cell support-ability of the pcECM was shown at much lower steady state values (5.4×10⁵ cells/cm², FIGS. 14A-14D). This value was surprisingly similar to that measured for endothelial cell density at the luminal side of the porcine lateral artery descending coronary artery (5.0±0.7×10⁵ cells/cm², FIGS. 14A-14D) as assessed through image analyses.

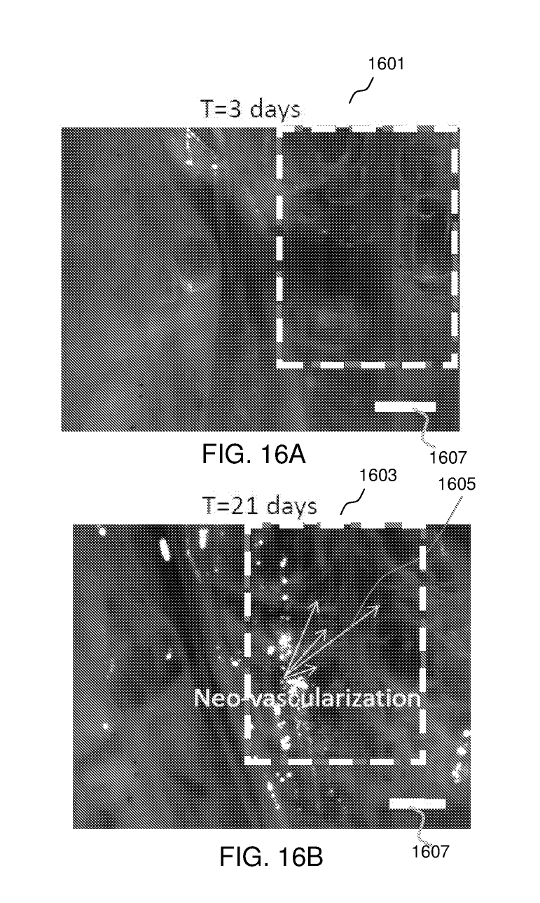

Finally, the possibility to promote neo-vascularization ex vivo was demonstrated using the dynamic co-cultivation of the hMSC and HUVECs in the bioreactor setup. This was evident by bright field images taken from the same location at sequential time points (FIG. 16A-16B). New blood vascular structures appeared to form and connect to previously established larger conduits within the three weeks of the experiment.

The specific binding of the collagen-binding peptides (CBPs) to the decellularized thick pcECM was similar to that observed with commercial collagen thereby validating the pcECM structural integrity and suggesting that it may provide functional support for reseeded cells. Treating the pcECM with HA was shown to be more effective in increasing cell attachment and proliferation than treatments with RGD (Arg-Gly-Asp) containing collagen-binding peptides, cross-linking, nitrocellulose or sulfated glycosaminoglycans (GAGs).

Graph 1101 in FIG. 11A shows a comparison of the least square mean cell density (two-way ANOVA with Tukey's HSD post-hoc correction for interaction and α-level adjustment for multiple comparisons, n=6 per group) to test the statistical significance of differences among groups through time. Cell viability upon treated matrices was measured using the AlamarBlue™ assay at different time points post seeding. Graph 1101 in FIG. 11A shows two representative time-curves. EDC-NHS combined with hyaluronic acid treatment (EDC-NHS-HA) is represented by stars, and non-treated control pcECM is represented by triangle. As shown, EDC-NHS-HA yielded the best result in terms of cell proliferation. Table 1105 in FIG. 11B denotes statistical significance groups at α=0.05 by capital letters. Photograph 1107 in FIG. 11C shows H&E staining 16 days post seeding which reveals that cells were aligned along the ECM fibers.

To measure the scaffold's cell-holding capacity, increasing densities of hMSCs (n=5 per seeding density) were seeded on untreated and HA-treated decellularized scaffolds, cultured under static conditions for 24 h, and analyzed by AlamarBlue. The cell-loading capacity of HA-treated scaffolds (4.0×10⁵ cells/cm²) was significantly higher (p<0.0001) than that of the untreated scaffolds (2.7×10⁵ cells/cm²) (FIG. 6A), and demonstrated high correlation (R²>0.96) between the modeled and empirically measured cell attachments (FIG. 6B). To characterize the effect of cell attachment on the proliferation and cell growth profile, hMSCs were seeded on untreated and HA-treated scaffolds in two densities, one below the maximal density of untreated scaffolds (FIG. 6C) and one above the maximal density of HA-treated scaffolds (FIG. 6D), and cultured for 21 days. While no significant difference was observed between the treated matrices and pcECM control at the low seeding density, the HA-treated scaffolds of the high seeding density, supported cell growth in significantly higher densities (p<0.05) throughout most of the culturing period (14 days), finally approaching densities similar to those measured on the untreated scaffolds (1.8±0.3×10⁵ cells/cm²) after 21 days.

To assess the effect of the medium volume on the final density of the cultivated cells, hMSCs seeded on untreated scaffolds were cultured for 21 days in either 2 or 10 mL of excess culture media, showing higher final densities (3.1±0.8×10⁵ cells/cm², FIG. 6E) when excess volume was used corresponding to the predicted maximal cell capacity of the pcECM. Histological examination performed 21 days postseeding revealed that the cells were only able to penetrate 100 μm deep into the pcECM (FIG. 6F). Photograph 1301 of FIG. 13 shows further example of steady-state static culture penetration depth of hMSC on pcECM. Photograph 1301 shows histological cross sections counter stained with DAPI for hMSC nuclei. For each experiment, there are n=5 biological replicas. However, the volumetric density (˜2.7×10⁷ cells/cm³), estimated by dividing the surface density with cellular penetration depth of 0.01 cm/100 μm, was comparable to the reported density of cardiomyocytes (CM)—the myocardium resident parenchymal cells suggesting high pcECM supportability of physiological densities.

The same model was also applied for a different cell type, HUVECs, as an additional verification of the model sensitivity, revealing a pcECM maximal loading capacity for endothelial cells at a density of 5.4×10⁴ cells/cm². Graph 1401 in FIG. 14A shows a mathematical modeling of empirical data sets for HUVEC seeded on pcECM matrices. Graph 1403 in FIG. 14B shows a goodness-of-fit between predicted and measured values for HUVEC seeded on pcECM matrices. The HUVEC loading capacity of the pcECM scaffolds (5.4×10⁴ cells/cm²) was calculated to be five-fold lower than that measured for hMSCs (FIG. 6A-6B) on the same scaffolds. This difference can be attributed to different penetration depths as the HUVEC appeared to remain at a monolayer coating of the pcECM surface rather than penetrate inside and remodel it. Photograph 1405 of FIG. 14C shows H&E staining of representative histological cross-sections (14 days, static culture) which revealed that HUVECs form a monolayer coating on the pcECM surface and do not penetrate into it, hence lower cell densities are measured and predicted by the model. Scale bar 1409 in FIG. 14C represents 100 μm. Further, the values in FIGS. 14A and 14B for endothelial cells are also similar to the cell density of native porcine tissue coronary artery as evaluated through image analyses of confocal scans taken from within a freshly harvested porcine coronary artery (5.0±0.7×10⁴ cells/cm²). Photograph 1407 in FIG. 14D shows confocal image analyses of four region of interest (ROI) in at least three representative porcine coronary artery luminal longitudinal tile scans which resulted in similar endothelial density values (5.0±0.7×10⁴ cells/cm²). Scale bar 1411 in FIG. 14D represents 200 μm.

In the following, proving feasibility for pcECM support of tissue parenchymal cells (in this case cardiomyocytes) according to various embodiments will be provided.

The relevancy of pcECM to cardiac tissue engineering was demonstrated by its support of hESC-CM forming synchronously beating constructs just 3 days postseeding. Beating lasted for at least 3 more weeks during which time, histological cross sections revealed the presence of the hESC-CM, which were positively stained for Troponin I, serving as a marker of the cardiac muscle contraction machinery. In terms of maximal penetration depth, hESC-CMs were localized in the initial 100 μm distance from the surface, similar to what was observed with hMSC under identical static culture conditions.

In the following, compartmentalized recellularization according to various embodiments will be described.

A custom-made perfusion bioreactor (in other words a bioreactor module) 701 was designed and used (FIG. 7A) to study the ability of decellularized pcECM (FIG. 7B) to support compartmentalization of cell growth under dynamic culture conditions (FIG. 7). Simultaneous perfusion of two recellularized thick pcECM scaffolds revealed fully perfused constructs after 48 h that had regained their full dimensions (FIGS. 7C, 7D). Incubating bulk reseeded hMSCs for one and a half hours, before perfusion, yielded significantly higher (p<0.05) retention of cell densities (average of 92%±10% of the seeded cell quantity) compared to cells that were allowed to attach for 24 h, as determined following a day of perfusion at two physiologically relevant flow rates (40 and 80 mL/min, FIG. 7F). Utilizing an attachment time of 1.5 h followed by 7 days of perfusion at 40 mL/min (to decrease possible shear damages), resulted in hMSC penetration of up to 400 μm deep into the pcECM bulk (FIG. 7E, evidenced by cross-sectional H&E staining). Elongated cell nuclei aligned along the pcECM fibers suggest that cells were not only physically entrapped within the scaffold but also attached and anchored to the pcECM in a more natural way.

A second set of experiments (n=4) was performed to evaluate the long-term cell support ability of the dynamic culture system. HMSCs were statically precultured on the patch endocardial surface for 30 days, during which cell density steady states were reached (as modeled in FIGS. 6A-6F and imaged through live confocal in FIG. 7J). The subsequent dynamic cultivation for 14 days led to a significant increase (p<0.001) in cell proliferation of almost fourfold compared to the steady state value achieved under static conditions (FIG. 7G). Concomitantly, cell penetration toward the feeding blood vessels increased up to 13-fold compared to statically cultivated cells (FIGS. 6F and 7H, respectively). Immunofluorescent staining for CD44 indicated by 735, which may be green in colour, (counterstained with DAPI indicated by 731 which may be blue in colour, FIG. 7I) identified the reseeded cells attached through their HA receptor to the ECM fibers (autofluorescence indicated by 733, which may be red in colour).

From the above experiment, a method to cultivate tissue with increased cells penetration may be derived. According to various embodiments, the method may include seeding an exterior surface of a scaffold with a predetermined cell type, and supplying the scaffold with nutrients and oxygen from the other side or the interior. Such a scaffold may already have an inherent vascular network. The vessel of the inherent vascular network of the scaffold may be connected to an inlet 206 of a bioreactor module 200. The scaffold may be perfused via the inlet 206 of the bioreactor module 200 with culture medium to provide flow of nutrients and oxygen through the inherent vascular network of the scaffold to create a nutrient/oxygen gradient between the inherent vascular network and the surface of the scaffold to cause migratory diffusion induced penetration of cells towards the inherent vascular network. As a result, the seeded cells may penetrate deeper into the scaffold, towards the nutrient-rich and oxygen-rich regions of the scaffold. The scaffold may include a decelluralized extracellular matrix with an inherent vascular network preserved.

According to various embodiments, an in-vitro method for tissue cultivation may include seeding a surface of a scaffold with a predetermined cell type, and perfusing the scaffold from an opposite surface of the scaffold through the scaffold and towards the seeded surface with culture medium to provide flow of nutrients and oxygen through the scaffold to create a nutrient/oxygen gradient between the opposite surface and the seeded surface of the scaffold to cause migratory diffusion induced penetration of cells towards the opposite surface. The scaffold may include a scaffold containing an inherent vascular network. The method may further include connecting the vessel of the inherent vascular network of the scaffold to an inlet of the bioreactor module 200 as described herein such that the step of perfusing the scaffold may include perfusing via the inlet 206 of the bioreactor module 200 through the inherent vascular network of the scaffold

In another set of experiments, the applicability of this system to support the re-endothelialization of the pcECM vascular conduits was demonstrated. HUVECs stably expressing GFP appeared to form a “cobble stone-like” morphology, as assessed through confocal live imaging (FIG. 7K, 13 days postseeding and dynamic cultivation), achieving a monolayer coating of the vascular network lumen (FIG. 7I). Further immunofluorescent staining with CD31 performed on cross sections of dynamically re-endothelialized constructs confirmed the endothelial identity of the GFP-expressing cells and their retention as a monolayer without deviation to other compartments within the pcECM scaffold.

In the following, ex vivo assembly and functionality of the ECM vascular network according to various embodiments will be described.

The assembly and functionality of the vascular network were assessed using hMSCs and GFP-expressing HUVECs reseeded within different compartments of the pcECM (bulk injections and vasculature perfusion, respectively) and cocultured under dynamic conditions for 21 days. Online monitoring using indirect cellular viability and metabolism based assays revealed cell proliferation that was correlated to both increasing quantities of lactate production and to a parallel decrease in free glucose within the circulating culture media (FIGS. 8A, 8B, respectively). The addition of VEGF on day 14 substantially induced cell proliferation, which, a week later, reached a density of 3.0×10⁷ (±11%) cells per scaffold (FIG. 8A). Fluctuations in the measured concentrations from a baseline value to lower (glucose consumption) and higher (lactate production) levels are the natural result of culture media replenishing; however, the amplitudes of these fluctuations correspond to cell metabolism. The measured LDH levels were indicative of cell death in the early stages of cell seeding, revealing residual cell death in the matrix measured 3 days after HUVEC seeding and 2 days (t=9±1) after the hMSCs were added to the coculture. LDH levels stabilized with time to baseline levels, suggesting biocompatibility of the system that does not lead to any significant cytotoxic effect. The lactate levels measured exhibited physiological levels (2-8 mM, as per the lactate meter manufacturer instructions) throughout the entire experimental timeline.

The presence and organization of HUVEC-GFPs (t=3 and 21 days) was also monitored online by fluorescent microscopy (FIG. 8C-8E). Endothelial cells demonstrated sprouting of new capillary-like vessels either through preexisting pathways (FIG. 8E) or through the de novo ex vivo angiogenic sprouting (FIG. 9D). Confocal imaging of cross sections revealed that cocultured cells were able to reach an overall thickness of 1.7 mm (FIG. 9A). New blood vessels sprouted in areas containing high hMSC concentrations on the outer walls of preexisting blood vessels (indicted by rectangles FIG. 9A). The nascent blood vessel-like structures were observed as an “eruption” of endothelial cells 923 accompanied by hMSCs 925 (FIG. 9C, 9D).

In the following, a discussion of the experiments, the experimental results and the experimental data according to various embodiments will be presented.

Functional vascular supply is one of the most crucial impediments determining the post-transplantational fate of recellularized myocardial tissue constructs. Several strategies were suggested to circumvent these limitations. The use of cocultures incorporating endothelial and pericyte-like cells, with or without parenchymal model cells, was shown to improve the prospects of statically cultivated constructs by enhancing vessel sprouting and connectivity to the host tissue, post-transplantation. In another approach, dynamic cultivation in-vitro of nonvascularized constructs, using forced medium perfusion, was shown to increase cellular penetration and survival beyond diffusion limitations up to ˜600 μm from the surface. This value probably represents the upper bound of this approach, due to a tradeoff between insufficient supply of too-low perfusion pressures and excessive shear stress jeopardizing cell viabilities when too-high pressures are employed. In both these strategies, the key hurdle to achieving ultimate human applicable sized grafts is the long lag-time required for functional angiogenesis to occur (˜2-3 weeks postimplantation).

In recent years it is becoming clearer that “functional vascularization” is probably required to push the envelope of current tissue engineering technologies into cellularization of thicker and physiologically more relevant constructs. This is particularly true when the implantation site is ischemic, for example, the infarcted heart. In this context, the concept of “functional vascularization” is defined herein as the formation of a connectable branched vascular network within the construct that can be used to instantly supply the construct upon implantation. One approach to achieving such vascularization involves preimplantation of biomaterials either on the omentum or around femoral arteriovenous loops employed as cardiac surgical flaps with the aim of using the body as the ultimate supportive bioreactor. Another approach suggests the ex vivo construction of vascular beds from very basic building blocks using isolated native artery and vein embedded in a thymosin beta4-hydrogel. The functionality of this vascular bed and the ultimate cellularized tissue thicknesses that can be obtained by this approach are still not sufficiently understood. Though producing valuable insights, both the above approaches are associated with donor site morbidity, further complicating clinical applicability.

An alternative approach to attaining functional construct vascularization may be premised on the use of preserved vascular conduits within decellularized myocardial ECM. Indeed, procedures for isolating myocardial ECM of porcine origin have recently been reported—indicating the growing interest in this relatively new biomaterial. As the porcine heart is anatomically similar to the human heart, this thick composite bio-material holds high potential for myocardial replacement therapies. These scaffolds were also suggested to be advantageous over other materials given that they contain the ultra-structural mesh of inter-species conserved proteins and bioactive molecules that include natural myocardial ECM, which may better support expected regeneration and circumvent issues of immunogenicity. A distinction, however, should be made between whole heart porcine ECM templates and downscaled ventricular full-wall ECM scaffolds. Currently, the recellularization of big whole heart templates presents significant technological hurdles due to the complexity of cell types and quantities required, their effective delivery and organization within organ distinct parenchymal localities, and the development of relevant dynamic culturing technologies. The latter should enable continued viability and sterility for the relatively long time durations required for cell proliferation, organization, and maturation within their respective compartments. In this context the downscaling from whole heart templates to thick decellularized full wall ventricular slabs may be advantageous, pending sufficient preservation of the ventricular wall major ECM constituents and a supportive blood vessel infrastructure. Thus, downscaling will likely substantially reduce the complexity of cell quantities, types and delivery methods required for experimentation, and enable—under careful bioreactor system design (FIG. 3)—more feasible long-term experimentation with compartmentalized recellularization in a noncontaminated environment.

The study aimed to reconstruct an inherent functional vascular bed that supports recellularization of physiologically relevant thicknesses using a completely in-vitro setting (i.e., independent of donor organs or tissues). It was hypothesized that the preserved vascular network within the pcECM scaffold studied herein can provide the basis for the ex vivo construction of thick (i.e., >600 μm) recellularized myocardial-like tissue constructs, in a compartmentalized manner. For these purposes thick pcECM was evaluated herein in terms of its cell support capacity and functional vasculature assembly under static and dynamic culture conditions, using a bioreactor system custom-built to provide physiological mimicking conditions ex vivo. Two cell types were primarily employed in this study and used as model cells for endothelial (HUVECs) and pericytic/parenchymal (MSCs) functions to enable possible self-assembly of more mature and functional blood vessels within the construct. The technology developed herein may be readily applicable to many other ECM-based approaches, regardless of the ECM tissue origin or parenchymal cell types required for the engineered construct ultimate function, so long as the inherent vascular network is preserved within the material.

The cell supporting capacity of pcECM scaffolds was initially evaluated under static culture conditions. A simple methodology to mathematically model the maximal cell holding capacity of the pcECM (FIG. 6) was developed. The predicted (model) and measured (empirical data) maximal pcECM volumetric cell holding capacity (2.7×10⁷ cells/cm³) closely approximated the actual density of CM in the adult human heart (2×10⁷ CM/cm³). The suggested model was further validated in three ways. First, by artificially increasing the quantity of cell adhesion sites, a corresponding increase in the model's prediction of maximal cell capacity was revealed. Second, long-term cultivation exhibited convergence of cell densities even when the initial seeding densities were far below and above the model's estimated predictions. Third, when a different cell type was used (HUVECS) different values were obtained suggesting sensitivity of the model to specific cell-scaffold interactions (FIGS. 14A and 14B). Interestingly, the values measured and computed for HUVECs also corresponded to the measured endothelial cell density in the luminal surface of the porcine coronary artery and corresponded to that reported for completely biological engineered vascular grafts using human endothelial cells.

Assessment of luminal endothelial cell density in the porcine coronary arteries was conducted according to the following. Freshly isolated porcine hearts (n=3) were harvested from a local abattoir (Soon Hin Food Trading Pre. Ltd., Singapore). They were delivered to the lab on ice and immediately perfused with PFA 4% for 1 hr following which hearts were washed with cold PBS. A large slab (similar to the one cut for decellularization) containing the lateral anterior descending coronary artery (LAD) was cut from the heart, perfused and immersed in a DAPI staining solution for 20 min (NucBlue™, Life-Technologies, Singapore). The LAD were then opened from their epicardial side in a longitudinal artery cut. The exposed edges of the LAD were clamped to the sides and the slab was mounted on an inverted confocal microscope (LSM700, Carl Zeiss Germany) equipped with an EC Plan-Neofluar 10x/0.30 M27 air lens. Tile scan was performed for the DAPI signal containing at least a 3×3 fields of view per each artery. From each image, four regions of interest (ROI) were used for image analyses.

Several other mathematical models have been suggested in recent years to enable the characterization of cell quantities within scaffold biomaterials under both static and dynamic culture conditions. In general, the models based on static cultivation are usually too complex to be routinely used for biological screening or biomaterial characterizing, while the dynamic models are usually multifactorial, limiting applicability when simple static conditions are at hand. In this study, this is the first time a simple and easily applicable model may be suggested for these purposes and could probably be easily applied to any particular cell and biomaterial scaffold combination within 24-96 h of seeding. Furthermore, the findings indicate that maximal cell capacity is an important cell-scaffold characteristic, which may predict the scaffold's long-term cell support ability given a set of defined culture conditions. Nevertheless, this may be limited in applicability for scaffolds in which degradation is fast. For example, rapidly degrading scaffolds (e.g., PLGA) may modify their available surface area and diffusion patterns through time, affecting the cell maximal holding capacity of the scaffold. In this study, the pcECM was only mildly remodeled during the experimental timeline and therefore the findings remained valid for at least 2-3 weeks—a period of time, which is usually applicable for most of the practical laboratory tests.

As the pcECM scaffold was isolated using decellularization, some damage may be expected, which usually includes both GAG washout and the disruption of the collagenous structural network. GAG and collagen binding sites were previously reported to independently influence cell adhesion and proliferation within the heart. Therefore, screening experiments were performed in which cell adhesion sites were artificially introduced into the pcECM, with the aim of identifying the optimal cell adhesion site modification for model verification. These screening experiments may also indicate the extent of the damage that might have been caused by the decellularization, given that the addition of a missing component would be expected to result in increased cell attachment and proliferation on the pcECM. Thus, CBPs, GAG (representatives of both sulphated and nonsulfated groups), nitrocellulose, and simple crosslinking were evaluated with various combinations.

Thick pcECM matrices and commercial collagen were blocked with 5% bovine serum albumin (BSA) in PBS and labelled with Carboxy-tetramethylrhodamine (TAMRA) conjugated CBP. Photograph 1201 of FIG. 12A shows a labelled matrix which experienced a color change to pink-red. The color change was not diluted even after 10 consecutive washes compared to a non-treated control shown in photograph 1203 of FIG. 12A. Photograph 1205 of FIG. 12B shows fluorescent imagining of the crude labelled pcECM. Photograph 1207 of FIG. 12B shows fluorescent imaging of the commercial collagen serving as control. Photograph 1209 of FIG. 12C shows fluorescent imagining of cross cryo-sections taken out of labelled (14 ms exposure time). Photograph 1211 of FIG. 12D shows fluorescent imaging of non-labeled (5 s exposure time) matrix exhibiting a bright signal, suggesting peptide-target specific binding. Scale bars 1213 in FIGS. 12B, 12C and 12D represent 100 μm.

The functional collagen binding with specific CBP-RGD residues suggested the preservation of collagen-binding sites during the decellularization procedure. Furthermore, the fact that no significant difference was observed between pcECM treated with CBP containing RGD moieties compared to nontreated matrices in terms of cell attachment and proliferation suggests that structural binding motifs are not lacking within the pcECM. In contrast, addition of glycoside moieties (nitrocellulose, sulphated and nonsulfated GAG) were shown to be much better cell adhesion modifiers (with the best effect measured for HA addition, p=1.8×10⁻⁶) post decellularization. Interestingly, though initially differing, the final cell density (t=21) was similar in both HA-conjugated and nontreated pcECM matrices, suggesting that the effect of GAG conjugation is limited. This transient effect might be attributable to the synthesis and secretion of GAG by the reseeded cells themselves, altering the pcECM composition with time.

To better visualize the cells within the reseeded pcECM scaffolds, histological sections were performed, revealing a relatively high cell density within a narrow depth of penetration (˜100 μm). This limited penetration depth is similar to the known “soft-tissue” diffusion limitation that is associated with heart and muscle regeneration, further suggesting that the pcECM scaffolds can be recellularized to a physiologically similar density. Interestingly, the cultivation of cardiac parenchymal cells (i.e., CM) resulted in the formation of synchronously beating constructs starting from 3 days postseeding. In fact, the CMs were well supported by the pcECM within a similar penetration depth as that measured for hMSCs (FIGS. 15A and 15B).

It was therefore hypothesized that nutrient and medium volume may be a limiting factor in cell proliferation within these initial 100 μm of penetration depth. Increasing the volume of culture media per construct by fivefold enabled reaching the predicted theoretical values, for hMSCs used here as model cells, after 21 days of culture. However, no further improvements were observed, even when using larger medium volumes, pointing to the limitation associated with static culture conditions. This observation led to the development of the dynamic cultivation platform for thick pcECM constructs presented herein.

To enable long-term support of cellular proliferation a new perfusion bioreactor system was designed, encompassing physiological mimicking pulsatile perfusion capabilities along with room for electro-mechanical stimulation subsystems. It was hypothesized that by using the perfusion features of this bioreactor, cell support within a relatively thick and viable construct can be provided, which relies on the pcECM's inherently preserved blood vessel infrastructure. To test its applicability, a series of short-term optimizations were initially conducted to ensure proper physiological flow rates and sufficient seeding times were used. These experiments revealed that when hMSCs were injected into the bulk cavities of the thick pcECM scaffold and allowed to adhere for 90 min, high cell viabilities are achieved over 48 h periods, with reseeded and perfused constructs swelling back to human equivalent left ventricular dimensions. The cultivation of such constructs for 7 days revealed cell clusters aligned to the ECM fibers within the scaffold bulk, with a depth of up to four times greater than that observed under static culture conditions. Furthermore, the advantage of this bioreactor system over static tissue culture was demonstrated by sequential cultivation of statically precultured constructs that were allowed to reach cell density steady state, before their dynamic expansion for an additional period of 14 days. Under dynamic conditions, the observed increase in cell penetration and viability beyond the steady state values suggests that reseeded cells have migrated toward the nourishing blood vessels where presumably fresh oxygen and nutrients are available. The advantage of such an approach is in providing a biomimetic milieu for functional cell assembly that may, in turn, lead to CM orientation and survival.

Another major hurdle to any future transplantation strategy is achieving a proper vascular re-endothelialization of decellularized grafts, thus minimizing blood coagulation and aneurism formation. The re-endothelialization is also a prerequisite to the support of thick tissue-engineered constructs. As a proof of concept, the isolated thick pcECM scaffold and its adapted bioreactor system were also studied in terms of their ability to support such re-endothelialization. Seeing as both the coculture with MSC and the administration of VEGF were previously reported to be associated with blood vessel sprouting and maturation, they were both used in the experimental setup. Under dynamic coculture conditions, the blood vessel network of the thick pcECM constructs became revitalized, exhibiting various levels of vessel sprouting and maturation. MSCs added to the culture system demonstrated a pericytic-like support for the endothelial cells as evident by the histological cross-section analyses performed, and further supported by several reports on the role of MSCs as pericytes in vivo. The addition of VEGF to the culture media contributed to a dramatic increase in cell proliferation accompanied with vessel sprouting from various locations both through predetermined paths within the ECM and through de novo created tracks (FIGS. 8 and 9). FIGS. 16A and 16B show further example of neo vascularisation formed during dynamic cultivation. The dashed area in photograph 1601 of FIG. 16A shows a location of a specimen following 3 days of dynamic cultivation. The dashed area in photograph 1603 of FIG. 16B shows the same location of the same specimen following 21 days of dynamic cultivation. The arrows 1605 in photograph 1603 of FIG. 16B point to new-vessels which appeared to form and connect to pre-existing vessels. Scale bars 1607 in FIGS. 16A and 16B represent 2 mm.

The effect of VEGF addition to vessel sprouting was recently reported in a hydrogel model utilizing native arteries and veins as the main supplying vessels. Of note is that the effect of VEGF addition was time dependent as its addition in premature states (i.e., before MSC seeding) resulted in insignificant re-endothelialization (data not shown). This may be the first time such a delicate process of vessel sprouting from within large reseeded acellular conduits (<1 mm in diameter, ˜5-6 mm in length) has been documented in a completely in-vitro environment. Thus, the bioreactor and scaffold setup presented in this study may also be used for further studies of the delicate interplay between various cell types related to angiogenesis and cardiac restoration therapies.

Finally, the coculture of endothelial cells and MSCs using this novel perfusion bioreactor system also enabled the achievement of a relatively thick cell-supportive ECM tissue construct (˜1.7 mm), which is unprecedented in a completely in-vitro system. This is comparable to the maximal reported thickness achieved to date using in vivo corporal systems as the optimal bioreactor setup.

In conclusion, the study presents two major findings/methodologies: The ex vivo genesis of functional vasculature and the quick characterization of scaffold biomaterials in terms of their maximal cell capacity and long-term cell support ability. Both methodologies reported herein were demonstrated through the use of pcECM thick constructs and a new custom-developed dynamic cultivation technology. It has been shown that using such a careful and systematic approach, the support of physiological-like cell densities in up to 1.7 mm thick viable constructs is possible in a completely ex vivo environment. These findings may raise the bar for state-of-the-art myocardial tissue engineering and reaffirm the potential of thick acellular pcECM as an exciting biomaterial with a clinical potential for regenerative cardiac medicine. Furthermore, this bioreactor system offers a unique platform for in-vitro studies of decellularized soft-tissue ECM-based tissue engineering strategies, such as the pcECM demonstrated herein. Nevertheless, to achieve morphologies that better resemble the cardiac native tissue, the incorporation of parenchymal cells (e.g., CM) into the dynamically cultivated constructs and the study of additional mechano-electrical stimulation in the designed bioreactor, are required. Such experimentation may further exploit the potential of the thick pcECM matrix and bioreactor system reported herein.

According to various embodiments, the bioreactor module and the bioreactor system as substantially described herein may advantageously enable the production of functioning tissue for various types of soft tissue replacements, such as that of heart tissue. The scale of the tissue produced may be somewhere between a simple tissue and a whole organ, and could be adapted to fit human clinically relevant sized tissue slabs or engineered constructs.

Further, the bioreactor module and bioreactor system as substantially described herein may support simultaneously different cell types (e.g. pericytes and endothelial cells) within different niches of the tissue constructs, accomplishing a physiologically mimicking hierarchical organization. This capability is crucial for enabling native-tissue-like functionalities and has not been achieved to date using any reported technology.

Together with various embodiments of cultivation methodologies described herein, the bioreactor module and bioreactor system as substantially described herein may enable the cell-support and thick tissue formation for up to a few millimeters in depth. This may represent a major breakthrough and a crucial parameter towards achieving clinically relevant soft tissues in which tissue mass and thickness correspond to function. To date the reported thicknesses achieved in-vitro are limited by a few hundred micrometers (<600 μm). Furthermore, the incorporation of all three stimulations (i.e. perfusion, electrical and mechanical stimulation) using one functioning bioreactor as well as showing tissue supportability for over 21 days without jeopardizing sterility and cell viabilities using one functioning bioreactor is unprecedented.

According to various embodiments, the bioreactor module and the bioreactor system as substantially described herein may enable achieving complete physiological mimicking conditions ex vivo for the purpose of clinical TE and therapeutic RM. Any company in the biomedical sector may be interested in obtaining such a bioreactor module or bioreactor system. Establishment of the clinical benefits of employing the bioreactor module and the bioreactor system may be revolutionary to the field and may merit commercialization. Furthermore, the bioreactor module and the bioreactor system as described herein may not be limited to cardiac tissues and could be applied to other soft tissue constructs as well.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A bioreactor module comprising:
a container;
a holder removably receivable in the container, the holder adapted to hold a scaffold containing an inherent vascular network;
an inlet connectable to a vessel of the inherent vascular network of the scaffold;
an inflatable device disposed within the container, the inflatable device having a conduit extending through a wall of the container; and
a pair of electrodes attached to opposing walls of the container.

2. The bioreactor module as claimed in claim 1, further comprising an outlet in a wall of the container.

3. The bioreactor module as claimed in claim 1 or 2, wherein the inflatable device comprises a balloon, a tube or a diaphragm.

4. The bioreactor module as claimed in any of claims 1 to 3, further comprising a transparent window covering an opening of the container.

5. The bioreactor module as claimed in any of claims 1 to 4, wherein the holder comprises a pair of holders.

6. The bioreactor module as claimed in claim 5, wherein the container is adapted to receive the pair of holders so that the pair of holders is separated by a distance from each other, wherein the distance is variable.

7. The bioreactor module as claimed in any of claims 1 to 6, wherein the scaffold comprises a natural scaffold containing a natural inherent vascular network.

8. The bioreactor module as claimed in any of claims 1 to 6, wherein the scaffold comprises a synthetic scaffold containing an inherent vascular network formed in the synthetic scaffold.

9. The bioreactor module as claimed in any of claims 1 to 8, wherein an end of two or more vessels of the inherent vascular network of the scaffold is opened.

10. A bioreactor system comprising a bioreactor module as claimed in any of claims 1 to 9.

11. The bioreactor system as claimed in claim 10, further comprising a mechanical stimulation subsystem adapted to control the inflatable device of the bioreactor module to generate mechanical stimulation by controlling the inflation of the inflatable device.

12. The bioreactor system as claimed in claim 10 or 11, further comprising an electrical subsystem adapted to control the pair of electrodes of the bioreactor module to generate electrical pulses from the pair of electrodes.

13. The bioreactor system as claimed in claim 12, wherein the electrical pulses comprises custom designed electrical pulses.

14. The bioreactor system as claimed in any of claims 11 to 13, wherein the mechanical stimulation subsystem comprises:
a controller; and
an actuation mechanism adapted to inflate the inflatable device of the bioreactor module by pressurising the inflatable device based on instructions received from the controller.

15. The bioreactor system as claimed in claim 14, wherein the mechanical stimulation subsystem further comprises a feedback mechanism adapted to measure the pressure of the inflatable device.

16. The bioreactor system as claimed in claim 15, wherein the actuation mechanism comprises an actuator and a hydraulic pump adapted to supply pressurized fluid to the inflatable device.

17. The bioreactor system as claimed in claim 15, wherein the actuation mechanism comprises an actuator and a pneumatic pump adapted to supply pressurized air to the inflatable device.

18. The bioreactor system as claimed in claim 15, wherein the actuation mechanism comprises an actuator and a pneumatic pump adapted to supply pressurized gas to the inflatable device.

19. The bioreactor system as claimed in any of claims 15 to 18, wherein the feedback mechanism comprises a pressure transducer.

20. The bioreactor system as claimed in any of claims 12 to 19, wherein the electrical subsystem comprises a controller adapted to send electrical signals to the pair of electrodes of the bioreactor module to generate the electrical pulses.

21. The bioreactor system as claimed in claim 20, wherein the electrical pulses comprises tissue mimicking electrical wave form.

22. The bioreactor system as claimed in any of claims 10 to 21, further comprising:
a reservoir adapted to contain culture medium; and
a pump adapted to pump culture medium from the reservoir to the bioreactor module.

23. The bioreactor system as claimed in claim 22, further comprising an oxygenator and a no-return check valve located along a fluid communication between the pump and the bioreactor module to maintain a predetermined oxygen level in the culture medium.

24. The bioreactor system as claimed in any of claims 10 to 23, wherein the bioreactor module is located in an incubator.

25. The bioreactor system as claimed in claim 24, wherein the incubator is maintained at a predetermined temperature.

26. The bioreactor system as claimed in any of claims 22 to 25, further comprising a faucet located along a fluid communication from the bioreactor module.

27. The bioreactor system as claimed in any of claims 10 to 26, further comprising a return channel adapted to return culture medium from the bioreactor module to the reservoir.

28. An in-vitro method for tissue cultivation, comprising:
connecting a vessel of a inherent vascular network of a scaffold to an inlet of a bioreactor module as claimed in any of claims 1 to 9; and
perfusing the scaffold via the inlet of the bioreactor module.

29. An in-vitro method for tissue cultivation, comprising:
seeding an interior of a vessel of an inherent vascular network of a scaffold with a first cell type;
seeding an exterior surface of the scaffold with a second cell type; and
perfusing through the inherent vascular network of the scaffold with culture medium to facilitate compartmentalized co-cultivation of the first cell type and the second cell type in different niches of the tissue.

30. The in-vitro method as claimed in claim 29, wherein the perfusing through the inherent vascular network of the scaffold with culture medium comprises:
perfusing initially with a first culture medium; and
replacing the first culture medium with a second culture medium gradually to ensure cell acclimation to the culture media change towards co-culture conditions.

31. The in-vitro method as claimed in claim 30, wherein the first culture medium comprises M199 or Endothelial Growth Medium-2 medium.

32. The in-vitro method as claimed in claim 30 or 31, wherein the second culture medium comprises culture medium for supporting the co-culture conditions.

33. The in-vitro method as claimed in claim 32, wherein the second culture medium comprises alpha modified Eagle's medium or Endothelial Growth Medium-2 or mTEASER or Roswell Park Memorial Institute medium.

34. The in-vitro method as claimed in any of claims 29 to 33, further comprising adding growth factors and cytokines such as human recombinant vascular endothelial growth factor (VEGF) basic fibroblast growth factor or any other factor (cell type dependent) to the culture medium which diffusion can cause cell survival, proliferation, polarization, migration and integration.

35. The in-vitro method as claimed in any of claims 29 to 34, wherein seeding the interior of the vessel of an inherent vascular network of a scaffold comprise rotating the scaffold to coat the vessel with the first cell type.

36. The in-vitro method as claimed in any of claims 29 to 35, wherein seeding the exterior surface of the scaffold comprises seeding by injection into the surface.

37. The in-vitro method as claimed in any of claims 29 to 35, wherein seeding the exterior surface of the scaffold comprises seeding by pipettation on the surface.

38. The in-vitro method as claimed in any of claims 29 to 37, wherein the first cell type comprises endothelial cells and the second cell type comprises pericytic cells.

39. The in-vitro method as claimed in any of claims 29 to 38, wherein the first cell type comprises endothelial cells and the second cell type comprises parenchymal cells.

40. The in-vitro method as claimed in any of claims 29 to 39, wherein the scaffold comprises decelluralized extracellular matrix with an inherent vascular network preserved.

41. The in-vitro method as claimed in any of claims 29 to 40, further comprising connecting the vessel of the inherent vascular network of the scaffold to an inlet of a bioreactor module as claimed in any of claims 1 to 9, wherein perfusing through the inherent vascular network of the scaffold comprises perfusing via the inlet of the bioreactor module.

42. An in-vitro method for tissue cultivation, comprising:
seeding a surface of a scaffold with a predetermined cell type; and
perfusing the scaffold from an opposite surface of the scaffold through the scaffold and towards the seeded surface with culture medium to provide flow of nutrients and oxygen through the scaffold to create a nutrient/oxygen gradient between the opposite surface and the seeded surface of the scaffold to cause migratory diffusion induced penetration of cells towards the opposite surface.

43. The in-vitro method as claimed in claim 42, wherein the scaffold comprises a scaffold containing an inherent vascular network.

44. The in-vitro method as claimed in claim 43, wherein the scaffold comprises decelluralized extracellular matrix with an inherent vascular network preserved.

45. The in-vitro method as claimed in claim 42 or 44, further comprising connecting the vessel of the inherent vascular network of the scaffold to an inlet of a bioreactor module as claimed in any of claims 1 to 9, wherein perfusing the scaffold comprises perfusing via the inlet of the bioreactor module through the inherent vascular network.
